SEQUENCE LISTING (183 sequences)

SEQ ID NO: 1 - protein, 185 aa, Homo sapiens
MRRMRRMWATQGLAVALALSVLPGSRALRPGDCEVCISYLGRFYQDLKDRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASARTDL

SEQ ID NO: 2 - protein, 179 aa, Homo sapiens [sequence omitted]

SEQ ID NO: 3 - protein, 158 aa, Homo sapiens
LRPGDCEVCISYLGRFYQDLKDRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASARTDL

SEQ ID NO: 4 - protein, 159 aa, Homo sapiens
MLRPGDCEVCISYLGRFYQDLKDRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASARTDL

SEQ ID NO: 5 - protein, 187 aa, Homo sapiens
MWCASPVAVVAFCAGLLVSHPVLTQGQEAGGRPGADCEVCKEFLNRFYKSLIDRGVNFSLDTIEKELISFCLDTKGKENRLCYYLGATKDAATKILSEVTRPMSVHMPAMKICEKLKKLDSQICELKYEKTLDLASVDLRKMRVAELKQILHSWGEECRACAEKTDYVNLIQELAPKYAATHPKTEL

SEQ ID NO: 6 - protein, 161 aa, Homo sapiens
QEAGGRPGADCEVCKEFLNRFYKSLIDRGVNFSLDTIEKELISFCLDTKGKENRLCYYLGATKDAATKILSEVTRPMSVHMPAMKICEKLKKLDSQICELKYEKTLDLASVDLRKMRVAELKQILHSWGEECRACAEKTDYVNLIQELAPKYAATHPKTEL

SEQ ID NO: 7 - protein, 162 aa, Homo sapiens
MQEAGGRPGADCEVCKEFLNRFYKSLIDRGVNFSLDTIEKELISFCLDTKGKENRLCYYLGATKDAATKILSEVTRPMSVHMPAMKICEKLKKLDSQICELKYEKTLDLASVDLRKMRVAELKQILHSWGEECRACAEKTDYVNLIQELAPKYAATHPKTEL

SEQ ID NO: 8 - protein, 40 aa, Homo sapiens
ILDDWGETCKGCAEKSDYIRKINELMPKYAPKAASARTDL

SEQ ID NO: 9 - protein, 39 aa, Homo sapiens
LDDWGETCKGCAEKSDYIRKINELMPKYAPKAASARTDL

SEQ ID NO: 10 - protein, 38 aa, Homo sapiens
DDWGETCKGCAEKSDYIRKINELMPKYAPKAASARTDL

SEQ ID NO: 11 - protein, 37 aa, Homo sapiens
DWGETCKGCAEKSDYIRKINELMPKYAPKAASARTDL

SEQ ID NO: 12 - protein, 36 aa, Homo sapiens
WGETCKGCAEKSDYIRKINELMPKYAPKAASARTDL

SEQ ID NO: 13 - protein, 35 aa, Homo sapiens
GETCKGCAEKSDYIRKINELMPKYAPKAASARTDL

SEQ ID NO: 14 - protein, 34 aa, Homo sapiens
ETCKGCAEKSDYIRKINELMPKYAPKAASARTDL

SEQ ID NO: 15 - protein, 33 aa, Homo sapiens
TCKGCAEKSDYIRKINELMPKYAPKAASARTDL

SEQ ID NO: 16 - protein, 32 aa, Homo sapiens
CKGCAEKSDYIRKINELMPKYAPKAASARTDL

SEQ ID NO: 17 - protein, 15 aa, Homo sapiens
CKGCAEKSDYIRKIN

SEQ ID NO: 18 - protein, 15 aa, Homo sapiens
TCKGCAEKSDYIRKI

SEQ ID NO: 19 - protein, 15 aa, Homo sapiens
ETCKGCAEKSDYIRK

SEQ ID NO: 20 - protein, 15 aa, Homo sapiens
GETCKGCAEKSDYIR

SEQ ID NO: 21 - protein, 15 aa, Homo sapiens
WGETCKGCAEKSDYI

SEQ ID NO: 22 - protein, 15 aa, Homo sapiens
DWGETCKGCAEKSDY

SEQ ID NO: 23 - protein, 15 aa, Homo sapiens
DDWGETCKGCAEKSD

SEQ ID NO: 24 - protein, 15 aa, Homo sapiens
LDDWGETCKGCAEKS

SEQ ID NO: 25 - protein, 15 aa, Homo sapiens
ILDDWGETCKGCAEK

SEQ ID NO: 26 - protein, 15 aa, Homo sapiens
KILDDWGETCKGCAE

SEQ ID NO: 27 - protein, 15 aa, Homo sapiens
KKILDDWGETCKGCA

SEQ ID NO: 28 - protein, 15 aa, Homo sapiens
LKKILDDWGETCKGC

SEQ ID NO: 29 - protein, 14 aa, Homo sapiens
CKGCAEKSDYIRKI

SEQ ID NO: 30 - protein, 14 aa, Homo sapiens
TCKGCAEKSDYIRK

SEQ ID NO: 31 - protein, 14 aa, Homo sapiens
ETCKGCAEKSDYIR

SEQ ID NO: 32 - protein, 14 aa, Homo sapiens
GETCKGCAEKSDYI

SEQ ID NO: 33 - protein, 14 aa, Homo sapiens
WGETCKGCAEKSDY

SEQ ID NO: 34 - protein, 14 aa, Homo sapiens
DWGETCKGCAEKSD

SEQ ID NO: 35 - protein, 14 aa, Homo sapiens
DDWGETCKGCAEKS

SEQ ID NO: 36 - protein, 14 aa, Homo sapiens
LDDWGETCKGCAEK

SEQ ID NO: 37 - protein, 14 aa, Homo sapiens
ILDDWGETCKGCAE

SEQ ID NO: 38 - protein, 14 aa, Homo sapiens
KILDDWGETCKGCA

SEQ ID NO: 39 - protein, 14 aa, Homo sapiens
KKILDDWGETCKGC

SEQ ID NO: 40 - protein, 13 aa, Homo sapiens
CKGCAEKSDYIRK

SEQ ID NO: 41 - protein, 13 aa, Homo sapiens
TCKGCAEKSDYIR

SEQ ID NO: 42 - protein, 13 aa, Homo sapiens
ETCKGCAEKSDYI

SEQ ID NO: 43 - protein, 13 aa, Homo sapiens
GETCKGCAEKSDY

SEQ ID NO: 44 - protein, 13 aa, Homo sapiens
WGETCKGCAEKSD

SEQ ID NO: 45 - protein, 13 aa, Homo sapiens
DWGETCKGCAEKS

SEQ ID NO: 46 - protein, 13 aa, Homo sapiens
DDWGETCKGCAEK

SEQ ID NO: 47 - protein, 13 aa, Homo sapiens
LDDWGETCKGCAE

SEQ ID NO: 48 - protein, 13 aa, Homo sapiens
ILDDWGETCKGCA

SEQ ID NO: 49 - protein, 13 aa, Homo sapiens
KILDDWGETCKGC

SEQ ID NO: 50 - protein, 12 aa, Homo sapiens
CKGCAEKSDYIR

SEQ ID NO: 51 - protein, 12 aa, Homo sapiens
TCKGCAEKSDYI

SEQ ID NO: 52 - protein, 12 aa, Homo sapiens
ETCKGCAEKSDY

SEQ ID NO: 53 - protein, 12 aa, Homo sapiens
GETCKGCAEKSD

SEQ ID NO: 54 - protein, 12 aa, Homo sapiens
WGETCKGCAEKS

SEQ ID NO: 55 - protein, 12 aa, Homo sapiens
DWGETCKGCAEK

SEQ ID NO: 56 - protein, 12 aa, Homo sapiens
DDWGETCKGCAE

SEQ ID NO: 57 - protein, 12 aa, Homo sapiens
LDDWGETCKGCA

SEQ ID NO: 58 - protein, 12 aa, Homo sapiens
ILDDWGETCKGC

SEQ ID NO: 59 - protein, 11 aa, Homo sapiens
CKGCAEKSDYI

SEQ ID NO: 60 - protein, 11 aa, Homo sapiens
TCKGCAEKSDY

SEQ ID NO: 61 - protein, 11 aa, Homo sapiens
ETCKGCAEKSD

SEQ ID NO: 62 - protein, 11 aa, Homo sapiens
GETCKGCAEKS

SEQ ID NO: 63 - protein, 11 aa, Homo sapiens
WGETCKGCAEK

SEQ ID NO: 64 - protein, 11 aa, Homo sapiens
DWGETCKGCAE

SEQ ID NO: 65 - protein, 11 aa, Homo sapiens
DDWGETCKGCA

SEQ ID NO: 66 - protein, 11 aa, Homo sapiens
LDDWGETCKGC

SEQ ID NO: 67 - protein, 10 aa, Homo sapiens
CKGCAEKSDY

SEQ ID NO: 68 - protein, 10 aa, Homo sapiens
TCKGCAEKSD

SEQ ID NO: 69 - protein, 10 aa, Homo sapiens
ETCKGCAEKS

SEQ ID NO: 70 - protein, 10 aa, Homo sapiens
GETCKGCAEK

SEQ ID NO: 71 - protein, 10 aa, Homo sapiens
WGETCKGCAE

SEQ ID NO: 72 - protein, 10 aa, Homo sapiens
DWGETCKGCA

SEQ ID NO: 73 - protein, 10 aa, Homo sapiens
DDWGETCKGC

SEQ ID NO: 74 - protein, 9 aa, Homo sapiens
CKGCAEKSD

SEQ ID NO: 75 - protein, 9 aa, Homo sapiens
TCKGCAEKS

SEQ ID NO: 76 - protein, 9 aa, Homo sapiens
ETCKGCAEK

SEQ ID NO: 77 - protein, 9 aa, Homo sapiens
GETCKGCAE

SEQ ID NO: 78 - protein, 9 aa, Homo sapiens
WGETCKGCA

SEQ ID NO: 79 - protein, 9 aa, Homo sapiens
DWGETCKGC

SEQ ID NO: 80 - protein, 8 aa, Homo sapiens
CKGCAEKS

SEQ ID NO: 81 - protein, 8 aa, Homo sapiens
TCKGCAEK

SEQ ID NO: 82 - protein, 8 aa, Homo sapiens
ETCKGCAE

SEQ ID NO: 83 - protein, 8 aa, Homo sapiens
GETCKGCA

SEQ ID NO: 84 - protein, 8 aa, Homo sapiens
WGETCKGC

SEQ ID NO: 85 - protein, 7 aa, Homo sapiens
CKGCAEK

SEQ ID NO: 86 - protein, 7 aa, Homo sapiens
TCKGCAE

SEQ ID NO: 87 - protein, 7 aa, Homo sapiens
ETCKGCA

SEQ ID NO: 88 - protein, 7 aa, Homo sapiens
GETCKGC

SEQ ID NO: 89 - protein, 6 aa, Homo sapiens
CKGCAE

SEQ ID NO: 90 - protein, 6 aa, Homo sapiens
TCKGCA

SEQ ID NO: 91 - protein, 6 aa, Homo sapiens
ETCKGC

SEQ ID NO: 92 - protein, 5 aa, Homo sapiens
CKGCA

SEQ ID NO: 93 - protein, 5 aa, Homo sapiens
TCKGC

SEQ ID NO: 94 - protein, 4 aa, Homo sapiens
CKGC

SEQ ID NO: 95 - protein, 40 aa, Homo sapiens
KQILHSWGEECRACAEKTDYVNLIQELAPKYAATHPKTEL

SEQ ID NO: 96 - protein, 39 aa, Homo sapiens
QILHSWGEECRACAEKTDYVNLIQELAPKYAATHPKTEL

SEQ ID NO: 97 - protein, 38 aa, Homo sapiens
ILHSWGEECRACAEKTDYVNLIQELAPKYAATHPKTEL

SEQ ID NO: 98 - protein, 37 aa, Homo sapiens
LHSWGEECRACAEKTDYVNLIQELAPKYAATHPKTEL

SEQ ID NO: 99 - protein, 36 aa, Homo sapiens
HSWGEECRACAEKTDYVNLIQELAPKYAATHPKTEL

SEQ ID NO: 100 - protein, 35 aa, Homo sapiens
SWGEECRACAEKTDYVNLIQELAPKYAATHPKTEL

SEQ ID NO: 101 - protein, 34 aa, Homo sapiens
WGEECRACAEKTDYVNLIQELAPKYAATHPKTEL

SEQ ID NO: 102 - protein, 33 aa, Homo sapiens
GEECRACAEKTDYVNLIQELAPKYAATHPKTEL

SEQ ID NO: 103 - protein, 32 aa, Homo sapiens
EECRACAEKTDYVNLIQELAPKYAATHPKTEL

SEQ ID NO: 104 - protein, 31 aa, Homo sapiens
ECRACAEKTDYVNLIQELAPKYAATHPKTEL

SEQ ID NO: 105 - protein, 30 aa, Homo sapiens
CRACAEKTDYVNLIQELAPKYAATHPKTEL

SEQ ID NO: 106 - protein, 15 aa, Homo sapiens
LKQILHSWGEECRAC

SEQ ID NO: 107 - protein, 15 aa, Homo sapiens
KQILHSWGEECRACA

SEQ ID NO: 108 - protein, 15 aa, Homo sapiens
QILHSWGEECRACAE

SEQ ID NO: 109 - protein, 15 aa, Homo sapiens
ILHSWGEECRACAEK

SEQ ID NO: 110 - protein, 15 aa, Homo sapiens
LHSWGEECRACAEKT

SEQ ID NO: 111 - protein, 15 aa, Homo sapiens
HSWGEECRACAEKTD

SEQ ID NO: 112 - protein, 15 aa, Homo sapiens
SWGEECRACAEKTDY

SEQ ID NO: 113 - protein, 15 aa, Homo sapiens
WGEECRACAEKTDYV

SEQ ID NO: 114 - protein, 15 aa, Homo sapiens
GEECRACAEKTDYVN

SEQ ID NO: 115 - protein, 15 aa, Homo sapiens
EECRACAEKTDYVNL

SEQ ID NO: 116 - protein, 15 aa, Homo sapiens
ECRACAEKTDYVNLI

SEQ ID NO: 117 - protein, 15 aa, Homo sapiens
CRACAEKTDYVNLIQ

SEQ ID NO: 118 - protein, 14 aa, Homo sapiens
KQILHSWGEECRAC

SEQ ID NO: 119 - protein, 14 aa, Homo sapiens
QILHSWGEECRACA

SEQ ID NO: 120 - protein, 14 aa, Homo sapiens
ILHSWGEECRACAE

SEQ ID NO: 121 - protein, 14 aa, Homo sapiens
LHSWGEECRACAEK

SEQ ID NO: 122 - protein, 14 aa, Homo sapiens
HSWGEECRACAEKT

SEQ ID NO: 123 - protein, 14 aa, Homo sapiens
SWGEECRACAEKTD

SEQ ID NO: 124 - protein, 14 aa, Homo sapiens
WGEECRACAEKTDY

SEQ ID NO: 125 - protein, 14 aa, Homo sapiens
GEECRACAEKTDYV

SEQ ID NO: 126 - protein, 14 aa, Homo sapiens
EECRACAEKTDYVN

SEQ ID NO: 127 - protein, 14 aa, Homo sapiens
ECRACAEKTDYVNL

SEQ ID NO: 128 - protein, 14 aa, Homo sapiens
CRACAEKTDYVNLI

SEQ ID NO: 129 - protein, 13 aa, Homo sapiens
QILHSWGEECRAC

SEQ ID NO: 130 - protein, 13 aa, Homo sapiens
ILHSWGEECRACA

SEQ ID NO: 131 - protein, 13 aa, Homo sapiens
LHSWGEECRACAE

SEQ ID NO: 132 - protein, 13 aa, Homo sapiens
HSWGEECRACAEK

SEQ ID NO: 133 - protein, 13 aa, Homo sapiens
SWGEECRACAEKT

SEQ ID NO: 134 - protein, 13 aa, Homo sapiens
WGEECRACAEKTD

SEQ ID NO: 135 - protein, 13 aa, Homo sapiens
GEECRACAEKTDY

SEQ ID NO: 136 - protein, 13 aa, Homo sapiens
EECRACAEKTDYV

SEQ ID NO: 137 - protein, 13 aa, Homo sapiens
ECRACAEKTDYVN

SEQ ID NO: 138 - protein, 13 aa, Homo sapiens
CRACAEKTDYVNL

SEQ ID NO: 139 - protein, 12 aa, Homo sapiens
ILHSWGEECRAC

SEQ ID NO: 140 - protein, 12 aa, Homo sapiens
LHSWGEECRACA

SEQ ID NO: 141 - protein, 12 aa, Homo sapiens
HSWGEECRACAE

SEQ ID NO: 142 - protein, 12 aa, Homo sapiens
SWGEECRACAEK

SEQ ID NO: 143 - protein, 12 aa, Homo sapiens
WGEECRACAEKT

SEQ ID NO: 144 - protein, 12 aa, Homo sapiens
GEECRACAEKTD

SEQ ID NO: 145 - protein, 12 aa, Homo sapiens
EECRACAEKTDY

SEQ ID NO: 146 - protein, 12 aa, Homo sapiens
ECRACAEKTDYV

SEQ ID NO: 147 - protein, 12 aa, Homo sapiens
CRACAEKTDYVN

SEQ ID NO: 148 - protein, 11 aa, Homo sapiens
LHSWGEECRAC

SEQ ID NO: 149 - protein, 11 aa, Homo sapiens
HSWGEECRACA

SEQ ID NO: 150 - protein, 11 aa, Homo sapiens
SWGEECRACAE

SEQ ID NO: 151 - protein, 11 aa, Homo sapiens
WGEECRACAEK

SEQ ID NO: 152 - protein, 11 aa, Homo sapiens
GEECRACAEKT

SEQ ID NO: 153 - protein, 11 aa, Homo sapiens
EECRACAEKTD

SEQ ID NO: 154 - protein, 11 aa, Homo sapiens
ECRACAEKTDY

SEQ ID NO: 155 - protein, 11 aa, Homo sapiens
CRACAEKTDYV

SEQ ID NO: 156 - protein, 10 aa, Homo sapiens
HSWGEECRAC

SEQ ID NO: 157 - protein, 10 aa, Homo sapiens
SWGEECRACA

SEQ ID NO: 158 - protein, 10 aa, Homo sapiens
WGEECRACAE

SEQ ID NO: 159 - protein, 10 aa, Homo sapiens
GEECRACAEK

SEQ ID NO: 160 - protein, 10 aa, Homo sapiens
EECRACAEKT

SEQ ID NO: 161 - protein, 10 aa, Homo sapiens
ECRACAEKTD

SEQ ID NO: 162 - protein, 10 aa, Homo sapiens
CRACAEKTDY

SEQ ID NO: 163 - protein, 9 aa, Homo sapiens
SWGEECRAC

SEQ ID NO: 164 - protein, 9 aa, Homo sapiens
WGEECRACA

SEQ ID NO: 165 - protein, 9 aa, Homo sapiens
GEECRACAE

SEQ ID NO: 166 - protein, 9 aa, Homo sapiens
EECRACAEK

SEQ ID NO: 167 - protein, 9 aa, Homo sapiens
ECRACAEKT

SEQ ID NO: 168 - protein, 9 aa, Homo sapiens
CRACAEKTD

SEQ ID NO: 169 - protein, 8 aa, Homo sapiens
WGEECRAC

SEQ ID NO: 170 - protein, 8 aa, Homo sapiens
GEECRACA

SEQ ID NO: 171 - protein, 8 aa, Homo sapiens
EECRACAE

SEQ ID NO: 172 - protein, 8 aa, Homo sapiens
ECRACAEK

SEQ ID NO: 173 - protein, 8 aa, Homo sapiens
CRACAEKT

SEQ ID NO: 174 - protein, 7 aa, Homo sapiens
GEECRAC

SEQ ID NO: 175 - protein, 7 aa, Homo sapiens
EECRACA

SEQ ID NO: 176 - protein, 7 aa, Homo sapiens
ECRACAE

SEQ ID NO: 177 - protein, 7 aa, Homo sapiens
CRACAEK

SEQ ID NO: 178 - protein, 6 aa, Homo sapiens
EECRAC

SEQ ID NO: 179 - protein, 6 aa, Homo sapiens
ECRACA

SEQ ID NO: 180 - protein, 6 aa, Homo sapiens
CRACAE

SEQ ID NO: 181 - protein, 5 aa, Homo sapiens
ECRAC

SEQ ID NO: 182 - protein, 5 aa, Homo sapiens
CRACA

SEQ ID NO: 183 - protein, 4 aa, Homo sapiens
CRAC

(19) United States
(12) Patent Application Publication
(10) Pub. No.: US 2022/0111042 A1
(43) Pub. Date: Apr. 14, 2022

(54) MANF as a Regulator of Immune System Function

(71) Applicant: University of Massachusetts, Boston, MA (US)

(72) Inventor: Lawrence M. Schwartz, Pelham, MA (US)

(21) Appl. No.: 17/302,687

(22) Filed: May 10, 2021

Related U.S. Application Data
(63) Continuation of application No. 15/321,708, filed on Dec. 22, 2016, now abandoned.
(60) Provisional application No. 62/016,410, filed on Jun. 24, 2014.

(51) Int. Cl. A61K 39/39

(52) U.S. Cl. CPC A61K 39/39; A61K 2039/55516

(57) ABSTRACT

Methods and compositions for enhancing an immunological response by administering an immunogenic composition comprising an effective amount of a MANF family protein or fragment thereof and an antigen to a subject. Also, methods and compositions for decreasing an immune response by inhibiting or antagonizing the activity of a MANF family protein.

### CROSS-REFERENCE

This application claims the benefit of U.S. Provisional Application No. 62/016,410, filed Jun. 24, 2014, which application is incorporated herein by reference.

### BACKGROUND OF THE INVENTION

Most cells carry the genetic machinery needed to “commit suicide.” This process typically occurs by a program known as apoptosis. The ability to initiate apoptosis serves a variety of developmental and homeostatic roles in organisms. For example, a general rule of embryogenesis is that many more cells are produced within each lineage than are needed for organ formation. Local cell-cell interactions determine which ones are valuable members of the community and therefore should be retained, and which ones are surplus cells that should be selectively deleted. In some regions of the developing nervous system, this selection process can result in the death of up to 95% of the neurons before or shortly after birth. Apoptosis can also be employed to remove deleterious cells, such as self-reactive lymphocytes during negative selection in the thymus. Here too, more than 95% of the T cells that are produced in the animal die in the thymus because they have either failed to develop normally or instead recognize self-proteins as foreign.

While apoptosis provides a tool for building and maintaining the human body, its use comes at a high cost. It has been estimated that mis-regulation of apoptosis accounts for more than 70% of all human disease (Reed, 2006). Inappropriate activation of apoptosis can result in the loss of valuable, but condemned cells, such as neurons in Alzheimer's and Parkinson's Diseases. Conversely, some cells have genetic defects that prevent them from activating apoptosis under the appropriate conditions, which can allow them to persist and induce pathogenesis. This inhibition of apoptosis is what happens with most cancers and auto-immune diseases.

### SUMMARY OF THE INVENTION

In a first aspect, provide herein are methods of enhancing an immunological response, the methods comprising administering an immunogenic composition comprising an effective amount of a MANF family protein or fragment thereof and an antigen to a subject.

In some embodiments, the antigen is immunologically cross-reactive with a pathogen. In some embodiments, the pathogen is a virus. In some embodiments, the pathogen is a bacterium. In some embodiments, the pathogen is a parasite. In some embodiments, the pathogen is a fungus (including yeast).

In some embodiments, the antigen is immunologically cross-reactive with a cancer cell. In some embodiments, the cancer cell is a prostate cancer cell, a breast cancer cell, a colorectal cancer cell, a lung cancer cell, a pancreatic cancer cell, a renal cancer cell, a melanoma cancer cell, an ovarian cancer cell, a B-cell malignancy cell, a leukemia cell, a lymphoma cell, a neuroblastoma cell, a glioblastoma cell, a skin cancer cell, a liver cancer cell, a testicular cancer cell, an adrenal cancer cell, esophageal cancer cell, a sarcoma, a gastrointestinal cancer cell, a cervical cancer cell, a bone cancer cell, or a combination thereof. In some embodiments, the antigen is a tumor rejection antigen. In some embodiments, the tumor rejection antigen is a patient specific antigen, a tumor specific antigen, or a tissue-restricted antigen.

In a second aspect, provided herein are methods of treating cancer comprising administering an immunologic composition comprising an effective amount of a MANF family protein or fragment thereof and a tumor rejection antigen to a subject in need thereof.

In some embodiments, the tumor rejection antigen is a patient specific antigen. In some embodiments, the tumor rejection antigen is a tumor specific antigen. In some embodiments, the tumor rejection antigen is a tissue-restricted antigen.

In some embodiments, the subject in need thereof has breast cancer, colorectal cancer, lung cancer, pancreatic cancer, renal cancer, melanoma cancer, ovarian cancer, a B-cell malignancy, leukemia, lymphoma, a neuroblastoma, a glioblastoma, skin cancer, a liver cancer cell, a testicular cancer cell, an adrenal cancer cell, esophageal cancer cell, a sarcoma, a gastrointestinal cancer cell, a cervical cancer cell, a bone cancer cell or a combination thereof.

In some embodiments, the immunologic composition further comprises an aluminum based salt, a squalene-oil-water emulsion, Bacillus Calmette-Guerin (BCG), or a combination thereof.

In some embodiments, the immunologic composition is administered by injection. In some embodiments, the immunologic composition is administered by intranasal administration.

Some embodiments further comprise administering one or more booster compositions that comprise the antigen or tumor rejection antigen without the MANF family protein.

In some embodiments, the immunogenic composition is administered to the subject two or more times.

In a third aspect, provided herein are methods of treating cancer comprising administering to a subject in need thereof an effective amount of a MANF family protein or fragment thereof and genetically engineered T cells expressing a chimeric antigen receptor.

In some embodiments, the chimeric antigen receptor comprises an antigen recognition region and an endodomain. In some embodiments, the target antigen of the antigen recognition region is α-Folate receptor, CALX, CD19, CD20, CD22, CD30, CD33, CD44v7/8, CEA, EGP-2, EGP-40, erb-B2, erb-B 2,3,4, FBP, Fetal acetylcholine receptor, GD2, GD3, Her2/neu, IL-13R-a2, KDR, k-light chain, LeY, L1 cell adhesion molecule, MAGE-A1, Mesothelin, Murine CMV infected cells, MUC1, NKG2D ligands, Oncofetal antigen (h5T4), PSCA, PSMA, TAA targeted by mAb IgE, TAG-72, or VEGF-R2. In some embodiments, the endodomain comprises ScFv-FcεRIγCAIX, ScFv-FcεRIγ, ScFv-CD3ζ (EBV), ScFv-CD3ζ, ScFv-CD28-CD3ζ, CD3ζ(EBV), ScFv-CD28-CD3ζ, CD3ζ, ScFv-CD3ζ, ScFv-41BB-CD3ζ, ScFv-41BB-CD3ζ, ScFv-CD3ζ (Influenza MP-1), ScFv-CD3ζ (VZV), ScFv-CD4-CD3ζ, CD3 ζ/CD137/CD28, ScFv-CD28-41 BB-CD3ζ, ScFv-CD8-CD3ζ, ScFv-FceRIγ, CD28/4-1 BB-CD3ζ, ScFv-CD28-CD3ζ (Influenza), ScFv-CD28mut-CD3ζ, Heregulin-CD3ζ, ScFv-FcεRIγ (alloantigen), ScFv-CD28, ScFv-CD28-OX40-CD3ζ, ScFv-CD3ξ, IL-13-CD28-4-1BB-CD3ζ, IL-13-CD3ζ, ScFv-CD28-CD3ζ, ScFV-CD4-FcεRIγ, ScFV-CD28-FcεRIγ, Ly49H-CD3ζ, NKG2D-CD3ζ, ScFV-CD3ζ (vaccination), ScFv-b2c-CD3ζ, or FceRI-CD28-CD3ζ (+a-TAA IgE mAb).

In some embodiments, the genetically engineered T cells were grown in the presence of the MANF family protein or fragment thereof. In some embodiments, the genetically engineered T cells were primed with the MANF family protein or fragment thereof. In some embodiments, the genetically engineered T cells were primed for about 10 seconds to about 1 hour with the MANF family protein or fragment thereof.

In some embodiments, the effective amount of the MANF family protein or fragment thereof is sufficient to increase cell viability of the genetically engineered T cells.

In a fourth aspect, provided herein are methods of treating cancer comprising administering an effective amount of a MANF family protein or fragment thereof and a bispecific monoclonal antibody to a subject in need thereof.

In some embodiments, the bispecific antibody comprises a tumor antigen epitope and a cytotoxic cell epitope. In some embodiments, the tumor antigen epitope binds to a tumor antigen on a prostate cancer cell, a breast cancer cell, a colorectal cancer cell, a lung cancer cell, a pancreatic cancer cell, a renal cancer cell, a melanoma cancer cell, an ovarian cancer cell, a B-cell malignancy cell, a leukemia cell, a lymphoma cell, a neuroblastoma cell, a glioblastoma cell, a skin cancer cell, or a combination thereof. In some embodiments, the cytotoxic cell epitope binds to a cytotoxic cell antigen on a T lymphocyte, a macrophage, a natural killer cell, a dendritic cell, or a combination thereof.

In some embodiments, the effective amount of the MANF family protein or fragment thereof is sufficient to enhance the survivability of endogenous cytotoxic cells.

In some embodiments, the effective amount of the MANF family protein or fragment thereof is sufficient enhance an immune response against tumor cells.

In some embodiments, the MANF family protein or fragment thereof is administered with the bispecific monoclonal antibody. In some embodiments, the MANF family protein or fragment thereof is administered before the bispecific monoclonal antibody. In some embodiments, the MANF family protein or fragment thereof is administered after the bispecific monoclonal antibody.

In some embodiments, administration is by injection.

In a fifth aspect, provided herein are methods to increase survival of a bone marrow transplant comprising administering an effective amount of a MANF family protein or fragment thereof to a subject undergoing a bone marrow transplant, so as to increase the survival of the bone marrow transplant as compared to a bone marrow transplant in the absence of the MANF family protein.

In some embodiments, the MANF family protein or fragment thereof is administered to the subject prior to the bone marrow transplant. In some embodiments, the MANF family protein or fragment thereof is administered to the subject concurrently with the bone marrow transplant. In some embodiments, the MANF family protein or fragment thereof is administered to the subject following the bone marrow transplant. In some embodiments, the MANF family protein or fragment thereof is administered to the site of the bone marrow transplant in the subject.

Some embodiments further comprise contacting bone marrow cells of the bone marrow transplant with the MANF family protein or fragment thereof prior to the bone marrow transplant.

In a sixth aspect, provided herein are methods to increase survival of a bone marrow transplant comprising contacting allogenic or autologous bone marrow cells ex vivo with an effective amount of a MANF family protein or fragment thereof thereby increasing the survival of the bone marrow transplant as compared to a bone marrow transplant in the absence of the MANF family protein.

In a seventh aspect, provided herein are methods of treating at least one condition associated with chemotherapy or radiation therapy comprising administering to a subject in need thereof an effective amount of a MANF family protein or fragment thereof.

In some embodiments, the MANF family protein or fragment thereof is administered to healthy tissue surrounding a tumor.

In some embodiments, the MANF family protein or fragment thereof is administered prior to the chemotherapy or radiation therapy.

In some embodiments, the effective amount of the MANF family protein or fragment thereof is from about 0.001 mg/kg to about 45 mg/kg. In some embodiments, the effective amount of the MANF family protein or fragment thereof is about 0.1 mg/kg to about 45 mg/kg. In some embodiments, the effective amount of the MANF family protein or fragment thereof is about 1 μg-500 μg. In some embodiments, the effective amount of the MANF family protein is about 5 μg-250 μg.

In some embodiments, the MANF family protein or fragment thereof is mesencephalic astrocyte derived neurotrophic factor (MANF) or a fragment thereof. In some embodiments, the MANF or fragment thereof comprises a peptide sequence that has at least about 80% identity with SEQ ID NO:3. In some embodiments, the MANF or fragment thereof consists of a sequence listed in Table 3. In some embodiments, the MANF or fragment thereof is cell permeable.

In some embodiments, the MANF family protein or fragment thereof is conserved dopaminergic neurotrophic factor (CDNF) or a fragment thereof. In some embodiments, the CDNF or fragment thereof comprises a peptide sequence that has at least about 80% identity with SEQ ID NO:6. In some embodiments, the CDNF or fragment thereof consists of a peptide sequence listed in Table 4. In some embodiments, the CDNF or fragment thereof is cell permeable.

In an eight aspect, provided herein are methods to increase survival or reduce tumor burden/size in a subject that has cancer comprising administering to the a MANF family protein antagonist, wherein administration of the MANF family protein antagonist to the subject increases survival of the subject or reduces tumor burden/size in the subject, wherein the MANF family protein antagonist reduces the level or activity of a MANF family protein.

In a ninth aspect, provided herein are methods to sensitize cells to at least one chemotherapeutic agent comprising administering a MANF family protein antagonist to a subject who is or will be undergoing chemotherapy, so as to sensitize cells to the chemotherapy as compared to chemotherapy in the absence of the MANF family protein antagonist.

In a tenth aspect, provided herein are methods to target self-reactive immune cells comprising administering a MANF family protein antagonist to a subject in need thereof, wherein the MANF family protein antagonist reduces the level and/or activity of at least one MANF family protein, so as to treat an auto-immune disorder.

In an eleventh aspect, provided herein are methods to treat an immune-mediated inflammatory disease (IMID) comprising administering a MANF family protein antagonist to a subject in need thereof, wherein the MANF family protein antagonist reduces the level and/or activity of at least one MANF family protein, so as to treat an auto-immune disorder.

In some embodiments, the subject has multiple sclerosis, irritable bowel syndrome, lupus, myasthenia gravis, rheumatoid arthritis, Hashimoto's thyroiditis, Grave's disease, autoimmune hepatitis, Alopecia, Addison's disease, Psoriasis, autoimmune pancreatitis, Celiac disease, pernicious anemia, Still's disease, juvenile arthritis, Felty syndrome, relapsing polychondritis, Guillain-Barré syndrome, vasculitis, or Crohn's disease.

In some embodiments, the MANF family protein antagonist is an anti-MANF antibody.

In some embodiments, the MANF family protein antagonist is an anti-CDNF antibody.

In some embodiments, the MANF family protein antagonist is an siRNA directed against a MANF gene.

In some embodiments, the MANF family protein antagonist is an siRNA directed against a CDNF gene.

While the proteins that comprise the core “killing” machinery of apoptosis have been defined, much less is known about the signaling pathways that allow this process to be regulated in a cell-type specific manner. By selectively targeting these signaling molecules, therapies that activate or inhibit apoptosis in a lineage-specific manner can be developed. For example, the provision of selective growth factors could allow valuable, but condemned cells, such as dopaminergic neurons in Parkinson's disease, to survive the insults that lead to their inappropriate death during disease.

Pharmaceutical compositions, immunogenic and/or vaccine compositions, and related methods for modulating immune responses are provided.

In one embodiment, an immunogenic composition comprising a MANF family protein, an antigen and a pharmaceutically acceptable carrier is provided. One embodiment provides a method of raising at least one cellular immune response comprising administering to a subject an immunogenic composition as described herein. Another embodiment provides a method of eliciting or enhancing an immune response in a subject, comprising administering to the subject a composition as described herein. Another embodiment provides a method of administering a vaccine composition to a subject comprising administering to the subject a vaccine composition comprising as an adjuvant a MANF family protein.

One embodiment provides a method to increase efficacy of immunotherapy treatment comprising administering a MANF family protein to a subject that is or will be undergoing immunotherapy treatment, so as to increase the efficacy of the immunotherapy treatment as compared to immunotherapy treatment in the absence of the MANF family protein.

Another embodiment provides a method to increase survival or reduce tumor burden/size in a patient that has cancer comprising administering to a patient with cancer an antagonist of a MANF family protein, wherein administration of the antagonist of the MANF family protein to the patient increases survival of the patient or reduces tumor burden/size in the patient, wherein the antagonist reduces the level or activity of a MANF family protein.

One embodiment provides a method to increase survival of a bone marrow transplant comprising administering a MANF family protein to a subject, or allogenic or autologous cells ex vivo, undergoing a bone marrow transplant, so as to increase the survival of the bone marrow transplant as compared to a bone marrow transplant in the absence of the MANF family protein.

Another embodiment provides a method of treating at least one condition associated with chemotherapy and/or radiation therapy comprising administering to a subject in need thereof an effective amount of a MANF family protein. A further embodiment provides a method to protect cells during administration of a pro-apoptotic treatment, comprising administering to a subject in need thereof an effective amount of a MANF family protein, wherein the pro-apoptotic treatment is chemotherapy and/or radiation therapy. In one embodiment, an antagonist of a MANF protein, including an anti-MANF or CNDF antibody, can be delivered to tumor cells to allow for increased elimination of such cells by chemotherapy or increased elimination of those cells by the immune system. In one embodiment, a MANF protein can be delivered to the liver, heart, bone marrow, or intestine to reduce off target toxicities of chemotherapeutic drugs (off target toxicities can prevent attainment of therapeutic levels of chemotherapeutic drug). In one embodiment, MANF can improve transplantation of hematopoietic stem cells.

One embodiment provides a method to sensitize cells to at least one chemotherapeutic agent comprising administering an antagonist of a MANF family protein to a subject who is or will be undergoing chemotherapy, so as to sensitize cells to the chemotherapy as compared to chemotherapy in the absence of the antagonist of the MANF family protein.

Another embodiment provides a method to target self-reactive immune cells comprising administering an agent to a subject in need thereof, wherein the agent reduces the level and/or activity of at least one MANF family protein, so as to treat an auto-immune disorder. One embodiment provides a method to treat an immune-mediated inflammatory disease (IMID) comprising administering an agent to a subject in need thereof, wherein the agent reduces the level and/or activity of at least one MANF family protein, so as to treat an auto-immune disorder. In one embodiment, the auto-immune disorder is multiple sclerosis, irritable bowel syndrome, lupus, myasthenia gravis, rheumatoid arthritis, Hashimoto's thyroiditis, Grave's disease, autoimmune hepatitis, Alopecia, Addison's disease, Psoriasis, autoimmune pancreatitis, Celiac disease, pernicious anemia, Still's disease, juvenile arthritis, Felty syndrome, relapsing polychondritis, Guillain-Barré syndrome, vasculitis, or Crohn's disease. In one embodiment, the agent is an anti-MANF or anti-CDNF antibody or a siRNA.

In one embodiment, the MANF family protein comprises MANF, CDNF, biologically active fragments thereof or a peptide or having at least 80% identity thereto.

### INCORPORATION BY REFERENCE

All publications, patents, and patent applications mentioned in this specification are herein incorporated by reference to the same extent as if each individual publication, patent, or patent application was specifically and individually indicated to be incorporated by reference. In the event that a term incorporated by reference conflicts with a term defined herein, this specification shall control.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides immunohistochemical staining of a human lymph node with an anti-MANF antiserum. MANF is expressed in the non-germinal cells. (Data obtained from Human Protein Atlas (available at www.proteinatlas.org/ENSG00000145050//tissue/lymph+node)).

FIG. 2 provides immunohistochemical staining of a human tonsil with an anti-MANF antiserum. MANF is expressed in the non-germinal cells. (Data obtained from Human Protein Atlas (available at www.proteinatlas.org/ENSG00000145050//tissue/tonsil)).

FIG. 3 provides an analysis of MANF expression in plasma, germinal center, and memory B cells (mBCs) from mice immunized with the T-dependent antigen, NP-CGG. Bars represent transformed cell count, plotted against the left Y-axis; rectangles represent the percentile rank within the sample, plotted against the right Y-axis.

FIGS. 4 A & B provide an analysis of MANF expression during a ligand screen in male C57BL/6 mice B cells incubated with B-cell activating factor (A) or 65 nM CD40 (B). Bars represent transformed cell count, plotted against the left Y-axis; rectangles represent the percentile rank within the sample, plotted against the right Y-axis (indicated with arrowhead where overlapping with the bars).

FIG. 5 provides an ELISA analysis of anti-KLH specific IgM titers between mice injected with 2,4,6, Trinitrophenyl hapten conjugated Keyhole Limpet Hemocyanin (TNP-KLH) and either 200 μL of 1 μg/μL MANF (KLH-MANF; right) or phosphate buffered saline (KLH-PBS; left). P=0.0013, Student's t-test.

FIG. 6 provides an analysis of MANF expression in human CD4+ T cell populations representing 5 successive stages of differentiation: intrathymic T progenitors, double positive thymocytes, single positive thymocytes, naïve T cells from cord blood, and naïve T cells from adult blood. Bars represent transformed cell count, plotted against the left Y-axis; rectangles represent the percentile rank within the sample, plotted against the right Y-axis (indicated with arrowhead where overlapping with the bars).

FIG. 7 provides an analysis of MANF expression in a Mus musculus cytotoxic T cell line (CTLL-2) at various time points up to 24 hours following interleukin-2 cytokine (IL-2) stimulation. IL-2 regulates T cell proliferation and differentiation. Bars represent count, plotted against the left Y-axis; rectangles represent the percentile rank within the sample, plotted against the right Y-axis.

FIG. 8 provides an analysis of MANF expression in Homo sapiens Natural Killer (NK) T cells. Bars represent count, plotted against the left Y-axis; rectangles represent the percentile rank within the sample, plotted against the right Y-axis.

FIG. 9 provides a Western blot analysis of COS-1 cells (control) and the T cell hybrid D011.10 cells using an anti-MANF antiserum. The 26S proteasome subunit Trip-1 was used as a loading control. Molecular weight markers are in kDa.

FIGS. 10A-B provides an analysis of MANF's ability to protect DO 11.10 cells from cell death caused by the glucocorticoid dexamethasone. A dose-dependent analysis was used to determine the LD50 (˜24 nM) for the cells (A). DO 11.10 cells were pretreated for 24 hrs with MANF (dose shown; B) and then challenged with 100 nM dexamethasone to induce cell death. *p=0.003; **p=0.019.

FIGS. 11A-B provide an analysis of MANF's ability to protect DO 11.10 cells from cell death caused by the DNA damaging chemotherapeutic drug cytosine arabinoside (Ara-C). A dose-response analysis was used to determine the LD50 for the cells (LD50=5 uM Ara-C) (A). A dose of 10 uM Ara-C was then used to treat DO 11.10 cells that had been pre-treated for 24 hrs with MANF (dose show; B). MANF conferred dose-dependent protection between 1-100 ng/mL.

FIG. 12 provides an analysis of MANF expression in Mus musculus splenocytes upon exposure to cyclophosphamide. Bars represent the log 2 ratio, plotted against the left Y-axis; rectangles represent the percentile rank within the sample, plotted on the right Y-axis.

FIG. 13 provides an analysis of MANF expression in colon tissue from RAG-1−/−C57BL/6 male mice (Mus musculus) injected with CD4+CD45RBhigh T cells from healthy wild type C57BL/6 males to induce colitis. T cells critically regulate clinical inflammatory bowel diseases (IBD). Bars represent the count, plotted against the left Y-axis; rectangles represent the percentile rank within the sample, plotted on the right Y-axis.

FIG. 14 provides an analysis of MANF expression in immortalized B cells from 60 unrelated human individuals (Homo sapiens) treated in vitro with tunicamycin to induce ER stress (left) or untreated as control, (right). ER stress induces the unfolded protein response (UPR). Bars represent the transformed count, plotted against the left Y-axis; rectangles represent the percentile rank within the sample, plotted on the right Y-axis.

FIG. 15 provides an analysis of MANF expression in mast cells isolated from the pancreatic lymph nodes of 60 day old prediabetic BioBiobreeding (BB) DR lyp/lyp animals (Rattus norvegicus). The BB DR lyp/lyp strain is a model for type 1 diabetes mellitus (T1DM). Bars represent the count, plotted against the left Y-axis; rectangles represent the percentile rank within the sample, plotted on the right Y-axis.

FIG. 16 provides immunohistochemical staining of a human lymph node. CDNF is expressed in the light zone of the germinal center. (Data obtained from Human Protein Atlas (available at www.proteinatlas.org/ENSG00000185267//tissue/lymph+node#img).)

### DETAILED DESCRIPTION OF THE INVENTION

For the purposes of clarity and a concise description, features can be described herein as part of the same or separate embodiments; however it will be appreciated that the scope of the invention may include embodiments having combinations of all or some of the features described.

The terminology used herein is for the purpose of describing particular embodiments only and is not intended to be limiting of the invention.

As used herein and in the appended claims, the singular forms “a,” “an,” and “the” include plural reference unless the context clearly dictates otherwise. Thus, for example, a reference to “a compound,” “a cell,” “a nucleic acid” or “a polypeptide” includes a plurality of such compounds, cells, nucleic acids or polypeptides (for example, a solution of cells, nucleic acids or polypeptides, a suspension of cells, or a series of compounds, cell, nucleic acid or polypeptide preparations), and so forth.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the term “comprising.”

As used herein, the term “about” means plus or minus 10% of the indicated value. For example, about 100 means from 90 to 110.

All genes and gene products (including RNA and proteins), and their respective names, disclosed herein are intended to correspond to homologs from any species for which the compositions and methods disclosed herein are applicable. When a gene or gene product from a particular species is disclosed, it is understood that this disclosure is intended to be exemplary only and is not to be interpreted as a limitation unless the context in which it appears clearly indicates otherwise. For example, the genes and gene products disclosed herein, which in some embodiments relate to mammalian (including human) nucleic acid and/or amino acid sequences, are intended to encompass homologous and/or orthologous and/or paralogous genes and gene products from other animals including, but not limited to, other mammals, fish, reptiles, amphibians, birds, and other vertebrates.

As used herein, the terms “polypeptide,” “peptide,” and “protein” are equivalent and mutually interchangeable. They refer to any amino acid chain, including native peptides, degradation products, synthetically synthesized peptides, or recombinant peptides; and include any post-translational modifications thereto (for example phosphorylation or glycosylation). Polypeptides include modified peptides, which may have, for example, modifications rendering the peptides more stable or less immunogenic. Such modifications can include, but are not limited to, cyclization, N-terminus modification, C-terminus modification, peptide bond modification, backbone modification and residue modification. Acetylation—amidation of the termini of the peptide (e.g., N-terminal acetylation and C-terminal amidation) can increase the stability and cell permeability of the peptides.

As used herein, the term “fragment” refers to a portion of a compound. For example, when referring to a protein, a fragment is a plurality of consecutive amino acids comprising less than the entire length of the polypeptide.

The disclosure of a particular sequence should be understood as disclosure of all fragments of a sequence. A fragment of a sequence can be defined according to a percent length of a reference sequence (e.g., a reference protein or peptide sequence). For example, a fragment of a sequence (e.g., protein or peptide sequence) can have a length that is at least about 1%, 2%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the length of the reference sequence. In another example, a fragment of a sequence (e.g., protein or peptide sequence) can have a length that is at most about 1%, 2%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the length of the reference sequence. In another example, a fragment of a sequence (e.g., protein or peptide sequence) can have a length that is about 1-99%, 2-99%, 5-99%, 10-99%, 20-99%, 30-99%, 40-99%, 50-99%, 60-99%, 70-99%, 80-99%, 90-99%, 2-90%, 5-90%, 10-90%, 20-90%, 30-90%, 40-90%, 50-90%, 60-90%, 70-90%, 80-90%, 5-80%, 10-80%, 20-80%, 30-80%, 40-80%, 50-80%, 60-80%, 70-80%, 10-70%, 20-70%, 30-70%, 40-70%, 50-70%, 60-70%, 20-60%, 30-60%, 40-60%, 50-60%, 30-50%, 40-50%, or 30-40% of the length of the reference sequence. Fragments can also be defined as have a percent identity to a reference sequence; for example a fragment can have length that is less than the reference sequence and a percent identity of the reference sequence.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SUM J. Applied Math. 48: 1073.

The disclosure of any particular sequence herein should be interpreted as the disclosure of all sequences sharing a percent identity with the sequence. A sequence can be defined herein according to a percent identity with a reference sequence. For example, the sequence can have at least about: 50%, 55%, 60%, 65%, 70%, 75%, 80%, 85%, 90%, 95%, 96%, 97%, 98%, 99%, or 100% identity with the reference sequence. In another example, the sequence can have about: 50-60%, 50-75%, 50-80%, 50-85%, 50-90%, 50-95%, 50-97%, 50-99%, 50-100%, 60-75%, 60-80%, 60-85%, 60-90%, 60-95%, 60-97%, 60-99%, 60-100%, 75-80%, 75-85%, 75-90%, 75-95%, 75-97%, 75-99%, 75-100%, 80-85%, 80-90%, 80-95%, 80-97%, 80-99%, 80-100%, 85-90%, 85-95%, 85-97%, 85-99%, 85-100%, 90-95%, 90-97%, 90-99%, 90-100%, 95-97%, 95-99%, 95-100%, 97-99%, 97-100%, or 99-100% identity with the reference sequence. Such sequences can be called variants of the reference sequence.

A “variant” of a polypeptide comprises an amino acid sequence wherein one or more amino acid residues are inserted into, deleted from and/or substituted into the amino acid sequence relative to another polypeptide sequence. The substituted amino acid(s) can be conservative substitutions or non-conservative substitutions, depending upon the context. Variants include fusion proteins.

Conservative substitutions are substitutions of one amino acid with a chemically similar amino acid. The following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W).

In making changes to the peptides and proteins disclosed herein, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein or peptide can be considered in designing variants of a protein or peptide. Certain amino acids can be substituted for other amino acids having a similar hydropathic index or score and still retain a similar biological activity. In making changes based upon the hydropathic index, the substitution of amino acids whose hydropathic indices are within ±2, ±1, or ±0.5 are included.

The substitution of like amino acids can also be made effectively on the basis of hydrophilicity. In certain embodiments, the greatest local average hydrophilicity of a protein or peptide, as governed by the hydrophilicity of its adjacent amino acids, correlates with a biological property of the protein or peptide.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, the substitution of amino acids whose hydrophilicity values are within ±2, ±1, ±0.5 are included.

As used herein, the term “subject” refers to any animal (e.g., mammals, birds, reptiles, amphibians, fish), including, but not limited to, humans, non-human primates, rodents, and the like, which is to be the recipient of a particular treatment. Typically, the terms “subject” and “patient” may be used interchangeably herein in reference to a subject.

As used herein, the term “administering” refers to providing an amount of a chemical or biological compound or pharmaceutical composition to a subject. The chemical or biological compound can be administered alone, but may be administered with other compounds, excipients, fillers, binders, carriers or other vehicles selected based upon the chosen route of administration and standard pharmaceutical practice. Administration may be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; ear drops; sprays, including nasal sprays; other proteins and peptides; synthetic polymers; microspheres; nanoparticles; and the like.

The active ingredients (e.g., chemicals or biological compounds or pharmaceutical compositions) disclosed herein can also be included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. Moreover, the packaging material may be biologically inert or lack bioactivity, such as plastic polymers, silicone, etc. and may be processed internally by the subject without affecting the effectiveness of the active ingredient.

An “adjuvant” is a pharmacological or immunological active ingredient that modifies the effects of another active ingredient. For example, a vaccine adjuvant can be a pharmacological or immunological active ingredient that increases the effectiveness of the vaccine. The increased effectiveness can be, e.g., due to an increased production or titer of an antibody to an antigen. In another example, a cell therapy adjuvant can be a pharmacological or immunological active ingredient that increases the effectiveness of the cell therapy. The increased effectiveness can be, e.g., due to an increased viability of the cells used in the cell therapy.

The term “effective amount,” as applied to the active ingredient(s) (e.g., compound(s), biologies and pharmaceutical compositions) described herein, means the quantity necessary to render the desired therapeutic result. For example, an effective amount is a level effective to treat, cure, or alleviate the symptoms of a disorder for which the therapeutic compound, biologic or composition is being administered. In another example, an effective amount of an adjuvant for a vaccine is an amount effective to increase an immunologic response to the vaccine antigen (e.g., to increase a titer of an antibody specific for the vaccine antigen). In another example, an effective amount of an adjuvant for cell therapy is an amount effective to increase the viability or effectiveness of the injected cells or tissues. Amounts effective for the particular therapeutic goal sought will depend upon a variety of factors including the disorder being treated and its severity and/or stage of development/progression; the bioavailability, and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and like factors well known to one of skill in the relevant scientific art. Some variation in dosage can occur depending upon the condition of the subject being treated, and the physician or other individual administering treatment will, in any event, determine the appropriate dose for an individual patient.

As used herein, “disorder” refers to a disorder, disease or condition, or other departure from healthy or normal biological activity, and the terms can be used interchangeably. The terms would refer to any condition that impairs normal function. The condition may be caused by sporadic or heritable genetic abnormalities. The condition may also be caused by non-genetic abnormalities.

The condition may also be caused by injuries to a subject from environmental factors, such as, but not limited to, cutting, crushing, burning, piercing, stretching, shearing, injecting, or otherwise modifying a subject's cell(s), tissue(s), organ(s), system(s), or the like.

As used herein, “treatment” or “treating” refers to arresting or inhibiting, or attempting to arrest or inhibit, the development or progression of a disorder and/or causing, or attempting to cause, the reduction, suppression, regression, or remission of a disorder and/or a symptom thereof. Various clinical and scientific methodologies and assays can be used to assess the development or progression of a disorder, and similarly, various clinical and scientific methodologies and assays can be used to assess the reduction, regression, or remission of a disorder or its symptoms. Additionally, treatment can be applied to a subject or to a cell culture.

### Introduction

Data provided herein demonstrates that MANF plays a role in immune system function and therefore has a number of direct clinical and research applications. For example, MANF has general utility as an adjuvant in vaccines to make them more effective. In another example, MANF has protective effects upon T cells and MANF expression can be induced by exposure to cytotoxic drugs.

MANF Family Proteins

Neurotrophic factors are small proteins that are synthesized and released predominantly by glial cells that induce neurons to up-regulate survival programs that help protect the cells from apoptosis. One of these neurotrophic factors, mesencephalic astrocyte-derived neurotrophic factor (MANF (NM 006010 (mRNA); NP_006001 (protein); US Pub Appln No. 20090282495)), is an 18 kDa secreted protein. Conserved dopaminergic neurotrophic factor (CDNF (NM_001029954 (mRNA); NP_001025125 (protein)) is the second member of the MANF family of proteins to be discovered.

When tested in primary nigral neuron cell cultures, MANF protected these midbrain dopaminergic neurons from death (Petrova et al., 2003). MANF also enhances the survival of dopaminergic neurons in vivo in the rat 6-hydroxydopamine (6-OHDA) model of Parkinson's disease. 6-OHDA is a potent and specific dopaminergic neuron toxin and is used to create an aggressive model of Parkinson's disease. Injection of MANF into 6-OHDA treated rat brains resulted in a statistically significant reduction in the loss of dopaminergic neurons and reduced the behavioral symptoms associated with the disorder (Voutilainen et al., 2009). These data are particularly exciting because MANF can improve dopaminergic neuron survival and correct motor defects up to 4 weeks after 6-OHDA treatment, a time when tyrosine hydroxylase (TH) the main biomarker for dopaminergic neurons is no longer normally detectable (Fitzpatrick et al., 2005). Thus, MANF can protect certain neurons from insults that result in cell death.

MANF also refers to any MANF family protein or active fragments thereof. The MANF family protein can be MANF or CDNF. As used herein, MANF or CNDF peptide comprises a protein having 70, 80, 85, 90, 95, 96, 97, 98, 99, or 100% homology (or identity) with the sequence of human: MANF or CDNF. In some embodiments, active fragments of these proteins can include peptides with a length of about 4-40 amino acids; for example, about: 4-40, 4-35, 4-30, 4-25, 4-20, 4-15, 4-10, 5-40, 6-40, 7-40, 8-40, 5-35, 5-30, 5-25, 5-20, 5-15, 5-10, 6-35, 6-30, 6-25, 6-20, 6-15, 6-10, 7-35, 7-30, 7-25, 7-20, 7-15, 7-10, 8-35, 8-30, 8-25, 8-20, or 8-15 amino acids. For example, the peptide can consist of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids.

Either MANF or CDNF can be the pro-form, which contains a signal sequence, or the mature, secreted form in which the signal sequence is cleaved off.

MANF family proteins can be a pro-form of MANF or an active fragment thereof. For example, the peptide sequence of the MANF family protein can comprise or consist of a sequence that has at least about 80% identity with SEQ ID NO: 1. In another example, the peptide sequence of the MANF family protein can comprise or consist of a sequence that has at least about 90% identity with SEQ ID NO: 1. In another example, the peptide sequence of the MANF family protein can comprise or consist of a sequence that has at least about 95% identity with SEQ ID NO: 1. In another example, the peptide sequence of the MANF family protein can comprise or consist of a sequence that has at least about 97% identity with SEQ ID NO: 1. In another example, the peptide sequence of the MANF family protein can comprise or consist of a sequence that has 100% identity with SEQ ID NO: 1. In any of these examples, the MANF family protein can have a length that is at least about 5% the length of SEQ ID NO: 1. In any of these examples, the MANF family protein can have a length that is at least about 50% the length of SEQ ID NO: 1. In any of these examples, the MANF family protein can have a length that is at least about 80% the length of SEQ ID NO: 1. In any of these examples, the MANF family protein can have a length that is at least about 90% the length of SEQ ID NO: 1. In any of these examples, the MANF family protein can have a length that is the same length as SEQ ID NO: 1. The MANF family protein, in any of these examples can also have a maximum length. The maximum length can be, e.g., 100%, 90%, 80%, 70%, 60%, 50%, or 25% the length of SEQ ID NO: 1.

MANF family proteins can be a pro-form of MANF or an active fragment thereof. For example, the peptide sequence of the MANF family protein can comprise or consist of a sequence that has at least about 80% identity with SEQ ID NO: 2. In another example, the peptide sequence of the MANF family protein can comprise or consist of a sequence that has at least about 90% identity with SEQ ID NO: 2. In another example, the peptide sequence of the MANF family protein can comprise or consist of a sequence that has at least about 95% identity with SEQ ID NO: 2. In another example, the peptide sequence of the MANF family protein can comprise or consist of a sequence that has at least about 97% identity with SEQ ID NO: 2. In another example, the peptide sequence of the MANF family protein can comprise or consist of a sequence that has 100% identity with SEQ ID NO: 2. In any of these examples, the MANF family protein can have a length that is at least about 5% the length of SEQ ID NO: 2. In any of these examples, the MANF family protein can have a length that is at least about 50% the length of SEQ ID NO: 2. In any of these examples, the MANF family protein can have a length that is at least about 80% the length of SEQ ID NO: 2. In any of these examples, the MANF family protein can have a length that is at least about 90% the length of SEQ ID NO: 2. In any of these examples, the MANF family protein can have a length that is the same length as SEQ ID NO: 2. The MANF family protein, in any of these examples can also have a maximum length. The maximum length can be, e.g., 100%, 90%, 80%, 70%, 60%, 50%, or 25% the length of SEQ ID NO: 2.

MANF family proteins can be a mature or secreted form of MANF, or an active fragment thereof. For example, the peptide sequence of the MANF family protein can comprise or consist of a sequence that has at least about 80% identity with SEQ ID NO: 3. In another example, the peptide sequence of the MANF family protein can comprise or consist of a sequence that has at least about 90% identity with SEQ ID NO: 3. In another example, the peptide sequence of the MANF family protein can comprise or consist of a sequence that has at least about 95% identity with SEQ ID NO: 3. In another example, the peptide sequence of the MANF family protein can comprise or consist of a sequence that has at least about 97% identity with SEQ ID NO: 3. In another example, the peptide sequence of the MANF family protein can comprise or consist of a sequence that has 100% identity with SEQ ID NO: 3. In any of these examples, the MANF family protein can have a length that is at least about 5% the length of SEQ ID NO: 3. In any of these examples, the MANF family protein can have a length that is at least about 50% the length of SEQ ID NO: 3. In any of these examples, the MANF family protein can have a length that is at least about 80% the length of SEQ ID NO: 3. In any of these examples, the MANF family protein can have a length that is at least about 90% the length of SEQ ID NO: 3. In any of these examples, the MANF family protein can have a length that is the same length as SEQ ID NO: 3. The MANF family protein, in any of these examples can also have a maximum length. The maximum length can be, e.g., 100%, 90%, 80%, 70%, 60%, 50%, or 25% the length of SEQ ID NO: 3.

MANF family proteins can be a synthetic form of MANF, or an active fragment thereof. The synthetic form of MANF contains a non-natural N-terminal methionine. The N-terminal methionine can enable production of the synthetic form of MANF in cell lines lacking the post-translational modification machinery to process the pro-form of MANF to the secreted or mature form of MANF. For example, the peptide sequence of the MANF family protein can comprise or consist of a sequence that has at least about 80% identity with SEQ ID NO: 4. In another example, the peptide sequence of the MANF family protein can comprise or consist of a sequence that has at least about 90% identity with SEQ ID NO: 4. In another example, the peptide sequence of the MANF family protein can comprise or consist of a sequence that has at least about 95% identity with SEQ ID NO: 4. In another example, the peptide sequence of the MANF family protein can comprise or consist of a sequence that has at least about 97% identity with SEQ ID NO: 4. In another example, the peptide sequence of the MANF family protein can comprise or consist of a sequence that has 100% identity with SEQ ID NO: 4. In any of these examples, the MANF family protein can have a length that is at least about 5% the length of SEQ ID NO: 4. In any of these examples, the MANF family protein can have a length that is at least about 50% the length of SEQ ID NO: 4. In any of these examples, the MANF family protein can have a length that is at least about 80% the length of SEQ ID NO: 4. In any of these examples, the MANF family protein can have a length that is at least about 90% the length of SEQ ID NO: 4. In any of these examples, the MANF family protein can have a length that is the same length as SEQ ID NO: 4. The MANF family protein, in any of these examples can also have a maximum length. The maximum length can be, e.g., 100%, 90%, 80%, 70%, 60%, 50%, or 25% the length of SEQ ID NO: 4.

TABLE 1
Human MANF Protein Sequences

| SEQ | | ASCESSION | |
|---|---|---|---|
| ID | NAME | Number | SEQUENCE |
| SEQ | Human | NP_006001 | MRRMRRMWAT QGLAVALALS |
| ID | Pro-MANF | | VLPGSRALRP GDCEVCISYL |
| NO: 1 | | | GRFYQDLKDR DVTFSPATIE |
| | | | NELIKFCREA RGKENRLCYY |
| | | | IGATDDAATK IINEVSKPLA |
| | | | HHIPVEKICE KLKKKDSQIC |
| | | | ELKYDKQIDL STVDLKKLRV |
| | | | KELKKILDDW GETCKGCAEK |
| | | | SDYIRKINEL MPKYAPKAAS |
| | | | ARTDL |
| SEQ | Human | | MWATQGLAVA LALSVLPGSR |
| ID | Pro-MANF | | ALRPGDCEVC ISYLGRFYQD |
| NO: 2 | | | LKDRDVTFSP ATIENELIKF |
| | | | CREARGKENR LCYYIGATDD |
| | | | AATKIINEVS KPLAHHIPVE |
| | | | KICEKLKKKD SQICELKYDK |
| | | | QIDLSTVDLK KLRVKELKKI |
| | | | LDDWGETCKG CAEKSDYIRK |
| | | | INELMPKYAP KAASARTDL |
| SEQ | Human | | LRPGDCEVCI SYLGRFYQDL |
| ID | MANF | | KDRDVTFSPA TIENELIKFC |
| NO: 3 | (Secreted | | REARGKENRL CYYIGATDDA |
| | Form) | | ATKIINEVSK PLAHHIPVEK |
| | | | ICEKLKKKDS QICELKYDKQ |
| | | | IDLSTVDLKK LRVKELKKIL |
| | | | DDWGETCKGC AEKSDYIRKI |
| | | | NELMPKYAPK AASARTDL |
| SEQ | Human | | MLRPGDCEVC ISYLGRFYQD |
| ID | Synthetic | | LKDRDVTFSP ATIENELIKF |
| NO: 4 | MANF | | CREARGKENR LCYYIGATDD |
| | | | AATKIINEVS KPLAHHIPVE |
| | | | KICEKLKKKD SQICELKYDK |
| | | | QIDLSTVDLK KLRVKELKKI |
| | | | LDDWGETCKG CAEKSDYIRK |
| | | | INELMPKYAP KAASARTDL |

MANF family proteins can be a pro-form of CDNF, or an active fragment thereof. For example, the peptide sequence of the MANF family protein can comprise or consist of a sequence that has at least about 80% identity with SEQ ID NO: 5. In another example, the peptide sequence of the MANF family protein can comprise or consist of a sequence that has at least about 90% identity with SEQ ID NO: 5. In another example, the peptide sequence of the MANF family protein can comprise or consist of a sequence that has at least about 95% identity with SEQ ID NO: 5. In another example, the peptide sequence of the MANF family protein can comprise or consist of a sequence that has at least about 97% identity with SEQ ID NO: 5. In another example, the peptide sequence of the MANF family protein can comprise or consist of a sequence that has 100% identity with SEQ ID NO: 5. In any of these examples, the MANF family protein can have a length that is at least about 5% the length of SEQ ID NO: 5. In any of these examples, the MANF family protein can have a length that is at least about 50% the length of SEQ ID NO: 5. In any of these examples, the MANF family protein can have a length that is at least about 80% the length of SEQ ID NO: 5. In any of these examples, the MANF family protein can have a length that is at least about 90% the length of SEQ ID NO: 5. In any of these examples, the MANF family protein can have a length that is the same length as SEQ ID NO: 5. The MANF family protein, in any of these examples can also have a maximum length. The maximum length can be, e.g., 100%, 90%, 80%, 70%, 60%, 50%, or 25% the length of SEQ ID NO: 5.

MANF family proteins can be a mature or secreted form of CDNF, or an active fragment thereof. For example, the peptide sequence of the MANF family protein can comprise or consist of a sequence that has at least about 80% identity with SEQ ID NO: 6. In another example, the peptide sequence of the MANF family protein can comprise or consist of a sequence that has at least about 90% identity with SEQ ID NO: 6. In another example, the peptide sequence of the MANF family protein can comprise or consist of a sequence that has at least about 95% identity with SEQ ID NO: 6. In another example, the peptide sequence of the MANF family protein can comprise or consist of a sequence that has at least about 97% identity with SEQ ID NO: 6. In another example, the peptide sequence of the MANF family protein can comprise or consist of a sequence that has 100% identity with SEQ ID NO: 6. In any of these examples, the MANF family protein can have a length that is at least about 5% the length of SEQ ID NO: 6. In any of these examples, the MANF family protein can have a length that is at least about 50% the length of SEQ ID NO: 6. In any of these examples, the MANF family protein can have a length that is at least about 80% the length of SEQ ID NO: 6. In any of these examples, the MANF family protein can have a length that is at least about 90% the length of SEQ ID NO: 6. In any of these examples, the MANF family protein can have a length that is the same length as SEQ ID NO: 6. The MANF family protein, in any of these examples can also have a maximum length. The maximum length can be, e.g., 100%, 90%, 80%, 70%, 60%, 50%, or 25% the length of SEQ ID NO: 6.

MANF family proteins can be a synthetic form of CDNF, or an active fragment thereof. The synthetic form of CDNF contains a non-natural N-terminal methionine. The N-terminal methionine can enable production of the synthetic form of CDNF in cell lines lacking the post-translational modification machinery to process the pro-form of CDNF to the secreted or mature form of CDNF. For example, the peptide sequence of the MANF family protein can comprise or consist of a sequence that has at least about 80% identity with SEQ ID NO: 7. In another example, the peptide sequence of the MANF family protein can comprise or consist of a sequence that has at least about 90% identity with SEQ ID NO: 7. In another example, the peptide sequence of the MANF family protein can comprise or consist of a sequence that has at least about 95% identity with SEQ ID NO: 7. In another example, the peptide sequence of the MANF family protein can comprise or consist of a sequence that has at least about 97% identity with SEQ ID NO: 7. In another example, the peptide sequence of the MANF family protein can comprise or consist of a sequence that has 100% identity with SEQ ID NO: 7. In any of these examples, the MANF family protein can have a length that is at least about 5% the length of SEQ ID NO: 7. In any of these examples, the MANF family protein can have a length that is at least about 50% the length of SEQ ID NO: 7. In any of these examples, the MANF family protein can have a length that is at least about 80% the length of SEQ ID NO: 7. In any of these examples, the MANF family protein can have a length that is at least about 90% the length of SEQ ID NO: 7. In any of these examples, the MANF family protein can have a length that is the same length as SEQ ID NO: 7. The MANF family protein, in any of these examples can also have a maximum length. The maximum length can be, e.g., 100%, 90%, 80%, 70%, 60%, 50%, or 25% the length of SEQ ID NO: 7.

TABLE 2
Human CDNF Protein Sequences

| SEQ | | ASCESSION | |
|---|---|---|---|
| ID | NAME | Number | SEQUENCE |
| SEQ | Human | NP_001025125 | MWCASPVAVV AFCAGLLVSH |
| ID | CDNF | | PVLTQGQEAG GRPGADCEVC |
| NO: 5 | Precursor | | KEFLNRFYKS LIDRGVNFSL |
| | | | DTIEKELISF CLDTKGKENR |
| | | | LCYYLGATKD AATKILSEVT |
| | | | RPMSVHMPAM KICEKLKKLD |
| | | | SQICELKYEK TLDLASVDLR |
| | | | KMRVAELKQI LHSWGEECRA |
| | | | CAEKTDYVNL IQELAPKYAA |
| | | | THPKTEL |
| SEQ | Human | | QEAGGRPGAD CEVCKEFLNR |
| ID | CDNF | | FYKSLIDRGV NFSLDTIEKE |
| NO: 6 | (Mature) | | LISFCLDTKG KENRLCYYLG |
| | | | ATKDAATKIL SEVTRPMSVH |
| | | | MPAMKICEKL KKLDSQICEL |
| | | | KYEKTLDLAS VDLRKMRVAE |
| | | | LKQILHSWGE ECRACAEKTD |
| | | | YVNLIQELAP KYAATHPKTE L |
| SEQ | Human | | MQEAGGRPGA DCEVCKEFLN |
| ID | Synthetic | | RFYKSLIDRG VNFSLDTIEK |
| NO: 7 | CDNF | | ELISFCLDTK GKENRLCYYL |
| | | | GATKDAATKI LSEVTRPMSV |
| | | | HMPAMKICEK LKKLDSQICE |
| | | | LKYEKTLDLA SVDLRKMRVA |
| | | | ELKQILHSWG EECRACAEKT |
| | | | DYVNLIQELA PKYAATHPKT |
| | | | EL |

Active fragments of MANF or CDNF can include short peptides with a length of about 4-40 amino acids; for example, about: 4-40, 4-35, 4-30, 4-25, 4-20, 4-15, 4-10, 5, 6, 7-40, 8, 5-35, 5-30, 5-25, 5-20, 5-15, 5-10, 6-35, 6-30, 6-25, 6-20, 6-15, 6-10, 7-35, 7-30, 7-25, 7-20, 7-15, 7-10, 8-35, 8-30, 8-25, 8-20, or 8-15 amino acids. For example, the preferred peptides can consist of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. The peptides may comprise any of the naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine as well as non-conventional or modified amino acids. The peptide can have 70, 80, 85, 90, 95, 96, 97, 98, 99, or 100% homology (or identity) with the sequence of human CDNF or MANF protein. In some embodiments, the peptides comprise the sequence CXXC. In some embodiments, the peptides comprise the sequence CKGC (SEQ ID NO:94) or CRAC (SEQ ID NO: 183) (see, e.g., WO 2013/034805). These peptides can be cell permeable. Active fragments of MANF can include any of the short peptides disclosed in Table 3. Active fragments of CDNF can include any of the short peptides disclosed in Table 4.

TABLE 3
Short peptides of human MANF.

| SEQ ID NO | SEQUENCE |
|---|---|
| SEQ ID NO: 8 | ILDDWGETCKGCAEKSDYIRKINELMPKYAPKAA |
| | SARTDL |
| SEQ ID NO: 9 | LDDWGETCKGCAEKSDYIRKINELMPKYAPKAAS |
| | ARTDL |
| SEQ ID NO: 10 | DDWGETCKGCAEKSDYIRKINELMPKYAPKAASA |
| | RTDL |
| SEQ ID NO: 11 | DWGETCKGCAEKSDYIRKINELMPKYAPKAASAR |
| | TDL |
| SEQ ID NO: 12 | WGETCKGCAEKSDYIRKINELMPKYAPKAASART |
| | DL |
| SEQ ID NO: 13 | GETCKGCAEKSDYIRKINELMPKYAPKAASARTDL |
| SEQ ID NO: 14 | ETCKGCAEKSDYIRKINELMPKYAPKAASARTDL |
| SEQ ID NO: 15 | TCKGCAEKSDYIRKINELMPKYAPKAASARTDL |
| SEQ ID NO: 16 | CKGCAEKSDYIRKINELMPKYAPKAASARTDL |
| SEQ ID NO: 17 | CKGCAEKSDYIRKIN |
| SEQ ID NO: 18 | TCKGCAEKSDYIRKI |
| SEQ ID NO: 19 | ETCKGCAEKSDYIRK |
| SEQ ID NO: 20 | GETCKGCAEKSDYIR |
| SEQ ID NO: 21 | WGETCKGCAEKSDYI |
| SEQ ID NO: 22 | DWGETCKGCAEKSDY |
| SEQ ID NO: 23 | DDWGETCKGCAEKSD |
| SEQ ID NO: 24 | LDDWGETCKGCAEKS |
| SEQ ID NO: 25 | ILDDWGETCKGCAEK |
| SEQ ID NO: 26 | KILDDWGETCKGCAE |
| SEQ ID NO: 27 | KKILDDWGETCKGCA |
| SEQ ID NO: 28 | LKKILDDWGETCKGC |
| SEQ ID NO: 29 | CKGCAEKSDYIRKI |
| SEQ ID NO: 30 | TCKGCAEKSDYIRK |
| SEQ ID NO: 31 | ETCKGCAEKSDYIR |
| SEQ ID NO: 32 | GETCKGCAEKSDYI |
| SEQ ID NO: 33 | WGETCKGCAEKSDY |
| SEQ ID NO: 34 | DWGETCKGCAEKSD |
| SEQ ID NO: 35 | DDWGETCKGCAEKS |
| SEQ ID NO: 36 | LDDWGETCKGCAEK |
| SEQ ID NO: 37 | ILDDWGETCKGCAE |
| SEQ ID NO: 38 | KILDDWGETCKGCA |
| SEQ ID NO: 39 | KKILDDWGETCKGC |
| SEQ ID NO: 40 | CKGCAEKSDYIRK |
| SEQ ID NO: 41 | TCKGCAEKSDYIR |
| SEQ ID NO: 42 | ETCKGCAEKSDYI |
| SEQ ID NO: 43 | GETCKGCAEKSDY |
| SEQ ID NO: 44 | WGETCKGCAEKSD |
| SEQ ID NO: 45 | DWGETCKGCAEKS |
| SEQ ID NO: 46 | DDWGETCKGCAEK |
| SEQ ID NO: 47 | LDDWGETCKGCAE |
| SEQ ID NO: 48 | ILDDWGETCKGCA |
| SEQ ID NO: 49 | KILDDWGETCKGC |
| SEQ ID NO: 50 | CKGCAEKSDYIR |
| SEQ ID NO: 51 | TCKGCAEKSDYI |
| SEQ ID NO: 52 | ETCKGCAEKSDY |
| SEQ ID NO: 53 | GETCKGCAEKSD |
| SEQ ID NO: 54 | WGETCKGCAEKS |
| SEQ ID NO: 55 | DWGETCKGCAEK |
| SEQ ID NO: 56 | DDWGETCKGCAE |
| SEQ ID NO: 57 | LDDWGETCKGCA |
| SEQ ID NO: 58 | ILDDWGETCKGC |
| SEQ ID NO: 59 | CKGCAEKSDYI |
| SEQ ID NO: 60 | TCKGCAEKSDY |
| SEQ ID NO: 61 | ETCKGCAEKSD |
| SEQ ID NO: 62 | GETCKGCAEKS |
| SEQ ID NO: 63 | WGETCKGCAEK |
| SEQ ID NO: 64 | DWGETCKGCAE |
| SEQ ID NO: 65 | DDWGETCKGCA |
| SEQ ID NO: 66 | LDDWGETCKGC |
| SEQ ID NO: 67 | CKGCAEKSDY |
| SEQ ID NO: 68 | TCKGCAEKSD |
| SEQ ID NO: 69 | ETCKGCAEKS |
| SEQ ID NO: 70 | GETCKGCAEK |
| SEQ ID NO: 71 | WGETCKGCAE |
| SEQ ID NO: 72 | DWGETCKGCA |
| SEQ ID NO: 73 | DDWGETCKGC |
| SEQ ID NO: 74 | CKGCAEKSD |
| SEQ ID NO: 75 | TCKGCAEKS |
| SEQ ID NO: 76 | ETCKGCAEK |
| SEQ ID NO: 77 | GETCKGCAE |
| SEQ ID NO: 78 | WGETCKGCA |
| SEQ ID NO: 79 | DWGETCKGC |
| SEQ ID NO: 80 | CKGCAEKS |
| SEQ ID NO: 81 | TCKGCAEK |
| SEQ ID NO: 82 | ETCKGCAE |
| SEQ ID NO: 83 | GETCKGCA |
| SEQ ID NO: 84 | WGETCKGC |
| SEQ ID NO: 85 | CKGCAEK |
| SEQ ID NO: 86 | TCKGCAE |
| SEQ ID NO: 87 | ETCKGCA |
| SEQ ID NO: 88 | GETCKGC |
| SEQ ID NO: 89 | CKGCAE |
| SEQ ID NO: 90 | TCKGCA |
| SEQ ID NO: 91 | ETCKGC |
| SEQ ID NO: 92 | CKGCA |
| SEQ ID NO: 93 | TCKGC |
| SEQ ID NO: 94 | CKGC |

TABLE 4
Short peptides of human CDNF.

| SEQ ID NO | SEQUENCE |
|---|---|
| SEQ ID NO: 95 | KQILHSWGEECRACAEKTDYVNLIQELAPKYAA |
| | THPKTEL |
| SEQ ID NO: 96 | QILHSWGEECRACAEKTDYVNLIQELAPKYAAT |
| | HPKTEL |
| SEQ ID NO: 97 | ILHSWGEECRACAEKTDYVNLIQELAPKYAATH |
| | PKTEL |
| SEQ ID NO: 98 | LHSWGEECRACAEKTDYVNLIQELAPKYAATHP |
| | KTEL |
| SEQ ID NO: 99 | HSWGEECRACAEKTDYVNLIQELAPKYAATHPK |
| | TEL |
| SEQ ID NO: 100 | SWGEECRACAEKTDYVNLIQELAPKYAATHPKT |
| | EL |
| SEQ ID NO: 101 | WGEECRACAEKTDYVNLIQELAPKYAATHPKTEL |
| SEQ ID NO: 102 | GEECRACAEKTDYVNLIQELAPKYAATHPKTEL |
| SEQ ID NO: 103 | EECRACAEKTDYVNLIQELAPKYAATHPKTEL |
| SEQ ID NO: 104 | ECRACAEKTDYVNLIQELAPKYAATHPKTEL |
| SEQ ID NO: 105 | CRACAEKTDYVNLIQELAPKYAATHPKTEL |
| SEQ ID NO: 106 | LKQILHSWGEECRAC |
| SEQ ID NO: 107 | KQILHSWGEECRACA |
| SEQ ID NO: 108 | QILHSWGEECRACAE |
| SEQ ID NO: 109 | ILHSWGEECRACAEK |
| SEQ ID NO: 110 | LHSWGEECRACAEKT |
| SEQ ID NO: 111 | HSWGEECRACAEKTD |
| SEQ ID NO: 112 | SWGEECRACAEKTDY |
| SEQ ID NO: 113 | WGEECRACAEKTDYV |
| SEQ ID NO: 114 | GEECRACAEKTDYVN |
| SEQ ID NO: 115 | EECRACAEKTDYVNL |
| SEQ ID NO: 116 | ECRACAEKTDYVNLI |
| SEQ ID NO: 117 | CRACAEKTDYVNLIQ |
| SEQ ID NO: 118 | KQILHSWGEECRAC |
| SEQ ID NO: 119 | QILHSWGEECRACA |
| SEQ ID NO: 120 | ILHSWGEECRACAE |
| SEQ ID NO: 121 | LHSWGEECRACAEK |
| SEQ ID NO: 122 | HSWGEECRACAEKT |
| SEQ ID NO: 123 | SWGEECRACAEKTD |
| SEQ ID NO: 124 | WGEECRACAEKTDY |
| SEQ ID NO: 125 | GEECRACAEKTDYV |
| SEQ ID NO: 126 | EECRACAEKTDYVN |
| SEQ ID NO: 127 | ECRACAEKTDYVNL |
| SEQ ID NO: 128 | CRACAEKTDYVNLI |
| SEQ ID NO: 129 | QILHSWGEECRAC |
| SEQ ID NO: 130 | ILHSWGEECRACA |
| SEQ ID NO: 131 | LHSWGEECRACAE |
| SEQ ID NO: 132 | HSWGEECRACAEK |
| SEQ ID NO: 133 | SWGEECRACAEKT |
| SEQ ID NO: 134 | WGEECRACAEKTD |
| SEQ ID NO: 135 | GEECRACAEKTDY |
| SEQ ID NO: 136 | EECRACAEKTDYV |
| SEQ ID NO: 137 | ECRACAEKTDYVN |
| SEQ ID NO: 138 | CRACAEKTDYVNL |
| SEQ ID NO: 139 | ILHSWGEECRAC |
| SEQ ID NO: 140 | LHSWGEECRACA |
| SEQ ID NO: 141 | HSWGEECRACAE |
| SEQ ID NO: 142 | SWGEECRACAEK |
| SEQ ID NO: 143 | WGEECRACAEKT |
| SEQ ID NO: 144 | GEECRACAEKTD |
| SEQ ID NO: 145 | EECRACAEKTDY |
| SEQ ID NO: 146 | ECRACAEKTDYV |
| SEQ ID NO: 147 | CRACAEKTDYVN |
| SEQ ID NO: 148 | LHSWGEECRAC |
| SEQ ID NO: 149 | HSWGEECRACA |
| SEQ ID NO: 150 | SWGEECRACAE |
| SEQ ID NO: 151 | WGEECRACAEK |
| SEQ ID NO: 152 | GEECRACAEKT |
| SEQ ID NO: 153 | EECRACAEKTD |
| SEQ ID NO: 154 | ECRACAEKTDY |
| SEQ ID NO: 155 | CRACAEKTDYV |
| SEQ ID NO: 156 | HSWGEECRAC |
| SEQ ID NO: 157 | SWGEECRACA |
| SEQ ID NO: 158 | WGEECRACAE |
| SEQ ID NO: 159 | GEECRACAEK |
| SEQ ID NO: 160 | EECRACAEKT |
| SEQ ID NO: 161 | ECRACAEKTD |
| SEQ ID NO: 162 | CRACAEKTDY |
| SEQ ID NO: 163 | SWGEECRAC |
| SEQ ID NO: 164 | WGEECRACA |
| SEQ ID NO: 165 | GEECRACAE |
| SEQ ID NO: 166 | EECRACAEK |
| SEQ ID NO: 167 | ECRACAEKT |
| SEQ ID NO: 168 | CRACAEKTD |
| SEQ ID NO: 169 | WGEECRAC |
| SEQ ID NO: 170 | GEECRACA |
| SEQ ID NO: 171 | EECRACAE |
| SEQ ID NO: 172 | ECRACAEK |
| SEQ ID NO: 173 | CRACAEKT |
| SEQ ID NO: 174 | GEECRAC |
| SEQ ID NO: 175 | EECRACA |
| SEQ ID NO: 176 | ECRACAE |
| SEQ ID NO: 177 | CRACAEK |
| SEQ ID NO: 178 | EECRAC |
| SEQ ID NO: 179 | ECRACA |
| SEQ ID NO: 180 | CRACAE |
| SEQ ID NO: 181 | ECRAC |
| SEQ ID NO: 182 | CRACA |
| SEQ ID NO: 183 | CRAC |

The peptides can be conjugated to a detectable chemical or biochemical moiety such as a FITC-label. As used herein, a “detectable chemical or biochemical moiety” means a tag that exhibits an amino acid sequence or a detectable chemical or biochemical moiety for the purpose of facilitating detection of the peptide; such as a detectable molecule selected from among: a visible, fluorescent, chemiluminescent, or other detectable dye; an enzyme that is detectable in the presence of a substrate, e.g., an alkaline phosphatase with NBT plus BCIP or a peroxidase with a suitable substrate; a detectable protein, e.g., a green fluorescent protein. Preferably, the tag does not prevent or hinder the penetration of the peptide into the target cell.

Adjuvant

In some embodiments, a MANF family protein, or fragment thereof, is not used as an antigen, but rather as an adjuvant—an agent to improve the immune response. Immunologic adjuvants are added to immunogenic compositions or vaccines to stimulate the immune system's response to the target antigen, but generally do not in themselves confer immunity. Adjuvants can act in various ways in presenting an antigen to the immune system. Adjuvants can act as a depot for the antigen, presenting the antigen over a long period of time, thus maximizing the immune response before the body clears the antigen. Adjuvants can also act as an irritant that causes the body to recruit and amplify its immune response.

Adjuvants can be added to a vaccine or immunogenic composition to promote a more rapid, a more potent and/or more persistent immune response to the vaccine. Adjuvants can enhance the immune response, for example, by extending the presence of antigen in the blood, helping to absorb the antigen presenting cells antigen, activating macrophages and lymphocytes, enhancing T cell activity, enhancing B cell survival, enhancing subsequent immune response, and/or supporting the production of cytokines. They can also allow for a lower vaccine dosage. Adjuvants can also aid in stabilizing formulations of antigens.

MANF and/or CDNF can be used as an adjuvant during immunization to make vaccines/immunogenic compositions more effective. MANF and/or CDNF are also a useful adjuvant to enhance the efficacy of immunotherapy treatments, for example, cancer treatments, making immunotherapy more effective. Immunotherapy as used herein is the treatment of disease by inducing, enhancing, or suppressing an immune response. Immunotherapies designed to elicit or amplify an immune response are classified as activation immunotherapies (e.g., immunogenic compositions or vaccines), while immunotherapies that reduce or suppress are classified as suppression immunotherapies. An immunogenic composition stimulates an immune response in the subject to which it is administered. An example of an immunogenic composition is a vaccine.

Certain embodiments include compositions that contain, in addition to a MANF family protein, or fragment thereof, at least one co-adjuvant, which refers to a component of such compositions that has adjuvant activity, but that is other than MANF and/or CDNF. A co-adjuvant having such adjuvant activity includes a composition that, when administered to a subject such as a human (e.g., a human patient), a non-human primate, a mammal or another higher eukaryotic organism having a recognized immune system, is capable of altering (e.g., increasing or decreasing) the potency and/or longevity of an immune response. (See, e.g., Powell and Newman, “Vaccine design—The Subunit and Adjuvant Approach”, 1995, Plenum Press, New York) In some embodiments disclosed herein MANF and/or CDNF and a desired antigen, and optionally one or more co-adjuvants, may so alter, e.g., elicit or enhance, an immune response that is directed against the desired antigen which may be administered at the same time as MANF and/or CDNF or may be separated in time and/or space (e.g., at a different anatomic site) in its administration.

Accordingly, and as noted above, co-adjuvants include compositions other than MANF and/or CDNF that have adjuvant effects, such as saponins and saponin mimetics, including QS21 and QS21 mimetics, alum, plant alkaloids such as tomatine, detergents such as (but not limited to) saponin, polysorbate 80, Span 85 and stearyl tyrosine, one or more cytokines (e.g., GM-CSF, IL-2, IL-7, IL-12, TNF-alpha, IFN-gamma), an imidazoquinoline immune response modifier, and a double stem loop immune modifier (dSLIM).

Antigen

An antigen, for use in certain embodiments of the compositions and methods described herein employing MANF and/or CDNF (e.g., as an adjuvant), can be any target epitope, molecule (including a biomolecule), molecular complex (including molecular complexes that contain biomolecules), subcellular assembly, cell or tissue against which elicitation or enhancement of immunoreactivity in a subject is desired. The term antigen can refer to a polypeptide antigen of interest. However, antigen, as used herein, may also refer to a recombinant construct that encodes a polypeptide antigen of interest (e.g., an expression construct). In certain embodiments the antigen may be, or may be derived from, or may be immunologically cross-reactive with, an infectious pathogen and/or an epitope, biomolecule, cell or tissue that is associated with infection, cancer, autoimmune disease, allergy, asthma, or any other condition where stimulation of an antigen-specific immune response would be desirable or beneficial, including but not limited to Multiple Sclerosis (MS), Irritable Bowel Syndrome (IBD) or Crohn's disease.

In certain embodiments the compositions described herein contain an antigen capable of eliciting an immune response against a human or other mammalian pathogen, which antigen may include a composition derived from a virus such as from HIV-1, human herpes viruses, cytomegalovirus, Rotavirus, Epstein Barr virus, Varicella Zoster Virus, or from a hepatitis virus such as hepatitis B virus, hepatitis A virus, hepatitis C virus and hepatitis E virus, or from other viral pathogens, such as paramyxoviruses: Respiratory Syncytial virus, parainfluenza virus, measles virus, mumps virus, human papilloma viruses, flaviviruses (e.g., Yellow Fever Virus, Dengue Virus, Tick-borne encephalitis virus, Japanese Encephalitis Virus), Ebola, or Influenza virus.

In some embodiments the compositions contain an antigen capable of eliciting an immune response against a human or other mammalian pathogen, which antigen may include a composition derived from one or more bacterial pathogens such as Neisseria spp, including N. gonorrhea and N. meningitides, S. pyogenes, S. agalactiae, S. mutans, H. ducreyi, Moraxella spp, Bordetella spp, including B. pertussis, B. parapertussis and B. bronchiseptica; Mycobacterium spp., including M. tuberculosis, M. bovis, M. leprae, M. avium, M. paratuberculosis, M. smegmatis; Legionella spp, including L. pneumophila; Escherichia spp, including enterotoxic E. coli, enterohemorragic E. coli, enteropathogenic E. coli; Vibrio spp, including V. cholera; Shigella spp, including S. sonnei, S. dysenteriae, S. flexnerii; Yersinia spp, including Y. enterocolitica, Y. pestis, Y. pseudotuberculosis; Campylobacter spp, including C. jejuni and C. coli; Salmonella spp, including S. typhi, S. paratyphi, S. choleraesuis, S. enteritidis; Listeria spp., including L. monocytogenes; Helicobacter spp, including H. pylori; Pseudomonas spp, including P. aeruginosa; Staphylococcus spp., including S. aureus, S. epidermidis; Enterococcus spp., including E. faecalis, E. faecium; Clostridium spp., including C. tetani, C. botulinum, C. difficile; Bacillus spp., including B. anthracis; Corynebacterium spp., including C. diphtheriae; Borrelia spp., including B. burgdorferi, B. garinii, B. ajzelii, B. andersonii, B. hermsii; Ehrlichia spp., including E. equi and the agent of the Human Granulocytic Ehrlichiosis; Rickettsia spp, including R. rickettsii; Chlamydia spp. including C. trachomatis, C. pneumoniae, C. psittaci; Leptospira spp., including L. interrogans; Treponema spp., including T. pallidum, T. denticola, T. hyodysenteriae; or other bacterial pathogens.

In other embodiments the compositions contain an antigen capable of eliciting an immune response against a human or other mammalian pathogen, which antigen may include a composition derived from one or more parasites such as Plasmodium spp., including P. falciparum; Toxoplasma spp., including T. gondii; Entamoeba spp., including E. histolytica; Babesia spp., including B. microti; Trypanosoma spp., including T. cruzi; Giardia spp., including G. lamblia; Leishmania spp., including L. major; Pneumocystis spp., including P. carinii; Trichomonas spp., including T. vaginalis; or from a helminth capable of infecting a mammal, such as: (i) nematode infections (including, but not limited to, Enterobius vermicularis, Ascaris lumbricoides, Trichuris trichiura, Necator americanus, Ancylostoma duodenale, Wuchereria bancrofti, Brugia malayi, Onchocerca volvulus, Dracanculus medinensis, Trichinella spiralis, and Strongyloides stercoralis); (ii) trematode infections (including, but not limited to, Schistosoma mansoni, Schistosoma haematobium, Schistosoma japonicum, Schistosoma mekongi, Opisthorchis sinensis, Paragonimus sp, Fasciola hepatica, Fasciola magna, Fasciola gigantica); and (iii) cestode infections (including, but not limited to, Taenia saginata and Taenia solium). Certain embodiments may therefore contemplate vaccine compositions that include an antigen derived from Schisostoma spp., Schistosoma mansonii, Schistosoma haematobium, and/or Schistosoma japonicum, or derived from fungi, including yeast, such as Candida spp., including C. albicans; Cryptococcus spp., including C. neoformans.

Another embodiment provides compositions and methods of use that can include an antigen that is derived from a cancer cell, as may be useful for the immunotherapeutic treatment of cancers. For example, the adjuvant formulation finds utility with tumor rejection antigens such as those for prostate, breast, colorectal, lung, pancreatic, renal or melanoma cancers.

Compositions (including an antagonist of a MANF family protein, such as an anti-MANF or anti-CDNF antibody or siRNA) and methods according to several embodiments can also be used for the prophylaxis or therapy of autoimmune diseases (including, but not limited to, Crohn's disease, Multiple Sclerosis, IBS (ulcerative colitis), which include diseases, conditions or disorders wherein a host's or subject's immune system detrimentally mediates an immune response that is directed against “self” tissues, cells, biomolecules (e.g., peptides, polypeptides, proteins, glycoproteins, lipoproteins, proteolipids, lipids, glycolipids, nucleic acids such as RNA and DNA, oligosaccharides, polysaccharides, proteoglycans, glycosaminoglycans, or the like, and other molecular components of the subjects cells and tissues) or epitopes (e.g., specific immunologically defined recognition structures such as those recognized by an antibody variable region complementarity determining region (CDR) or by a T cell receptor CDR.

Compositions (including an antagonist of a MANF family protein, such as an anti-MANF or CDNF antibody or siRNA) and methods according to several embodiments can also be used for the prophylaxis or therapy for immune-mediated inflammatory disease (IMID). An immune-mediated inflammatory disease (IMID) is any of a group of conditions or diseases characterized by common inflammatory pathways leading to inflammation, and which may result from, or be triggered by, a dysregulation of the normal immune response (one underlying manifestation of immune dysregulation is the inappropriate activation of inflammatory cytokines, such as IL-12, IL-6 or TNF alpha, whose actions can lead to pathological consequences). IMIDs can cause end organ damage, and are associated with increased morbidity and/or mortality. IMIDs include, but are not limited to, ankylosing spondylitis (AS), rheumatoid arthritis, Crohn's disease, psoriasis, psoriatic arthritis, Systemic lupus erythematosus, Type 1 diabetes, Multiple sclerosis.

Recombinant Expression Construct

In one embodiment, the MANF and/or CDNF composition may contain at least one recombinant expression construct that comprises a promoter operably linked to a nucleic acid sequence encoding an antigen and/or MANF or CDNF. In some embodiments, the recombinant expression construct is present in a viral vector, such as an adenovirus, adeno-associated virus, herpesvirus, lentivirus, poxvirus or retrovirus vector. Compositions and methods for making and using such expression constructs and vectors are known in the art, for the expression of polypeptide antigens.

Immune Response

One embodiment provides a composition for altering (e.g., increasing or decreasing, for example, relative to a control) immune responses in a host capable of mounting an immune response. As will be known to persons having ordinary skill in the art, an immune response may be any active alteration of the immune status of a host, which may include any alteration in the structure or function of one or more tissues, organs, cells or molecules that participate in maintenance and/or regulation of host immune status. Typically, immune responses may be detected by any of a variety of well-known parameters, including but not limited to in vivo or in vitro determination of: soluble immunoglobulins or antibodies; soluble mediators such as cytokines, lymphokines, chemokines, hormones, growth factors and the like as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death); or any other criterion by which the presence of an immune response may be detected.

Immune responses may often be regarded, for instance, as discrimination between self and non-self structures by the cells and tissues of a host's immune system at the molecular and cellular levels, but the invention should not be so limited. For example, immune responses may also include immune system state changes that result from immune recognition of self-molecules, cells or tissues, as may accompany any number of normal conditions such as typical regulation of immune system components, or as may be present in pathological conditions such as the inappropriate autoimmune responses observed in autoimmune and degenerative diseases. As another example, in addition to induction by up-regulation of particular immune system activities (such as antibody and/or cytokine production, or activation of cell mediated immunity) immune responses may also include suppression, attenuation or any other down-regulation of detectable immunity.

Immunotherapy is the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Cancer immunotherapy attempts to stimulate the immune system to reject and destroy tumors. Immuno cell therapy for cancer was first introduced by Rosenberg and his colleagues from the National Institute of Health USA. In the late 1980s, they published an article in which they reported a low tumor regression rate (2.6-3.3%) in 1205 patients with metastatic cancer who underwent different types of active specific immunotherapy (ASI). Initially Immunotherapy treatments involved administration of cytokines such as Interleukin (e.g., IL-2, IL-7, IL-12). Thereafter the adverse effects of such intravenously administered cytokines lead to the extraction of the lymphocytes from the blood and expanding them in vitro against tumor antigen before injecting the cells back into the patient along with stimulatory cytokines. The cells can then specifically target and destroy the tumor expressing antigen against which they have been raised. Immunotherapies can also be used for other diseases, including by not limited to, autoimmune diseases, MS, IBS and Crohns. MANF/CDNF can be useful as an adjuvant to enhance the efficacy of immunotherapy treatments.

Cancer chemotherapy can be an effective tool in treating cancer. However, some tumor cells develop mechanisms of resistance to apoptosis and so don't respond to the majority of cytotoxic therapies. Sensitization, with use of various sensitizing agents, of tumor cells for chemotherapy, immunotherapy or radiotherapy can reverse tumor cell resistance to such therapies. Treatment would therefore be a combination of at least one cytotoxic (e.g., chemotherapy, immunotherapy or radiotherapy) and at least one sensitizing agent, such as an antagonist of MANF, e.g., an antibody directed to MANF, CDNF or an siRNA (including humanized anti-MANF antibodies; MANF antibodies are commercially available, see for example, Sigma, St. Louis, Mo. or LifeSpan BioSciences, Inc.).

Bone marrow transplants can be conducted to treat severe diseases of the bone marrow, including certain forms of cancer. In a bone marrow transplant, hematopoietic stem cells are removed from a person and infused into another person (allogenic) or into the same person at a later time (autologous). If the donor and recipient are compatible, these infused cells will then travel to the bone marrow and initiate blood cell production. Transplantation from one person to another is conducted for the treatment of severe bone marrow diseases, such as congenital defects, autoimmune diseases or malignancies. Administration of MANF and/or CDNF before, after or during bone marrow transplantation can enhance survival of the bone marrow transplant.

Pharmaceutical Compositions

The active ingredients can be provided in a pharmaceutical composition. The pharmaceutical composition can comprise pharmaceutically acceptable diluent(s), excipient(s), or carriers). The pharmaceutical compositions can include other medicinal or pharmaceutical agents, carriers, adjuvants, such as preserving, stabilizing, wetting or emulsifying agents, solution promoters, salts for regulating the osmotic pressure, and/or buffers. Methods well known in the art for making formulations are to be found in, for example, Remington: The Science and Practice of Pharmacy, (20th ed.) ed. A. R. Gennaro A R., 2000, Lippencott Williams & Wilkins.

Pharmaceutical compositions generally comprise MANF and or CDNF, and may further comprise one or more components as provided herein that are selected, for example, from antigen, co-adjuvant, and/or a recombinant expression construct, in combination with a pharmaceutically acceptable carrier, excipient or diluent.

One embodiment provides MANF or CDNF “monotherapy” wherein MANF or CDNF is formulated in a composition that is substantially devoid of other antigens, and is administered to a subject in order to treat or prevent a disease or other condition (e.g., cancer, immunotherapy, targeted MANF/CDNF treatment to protect cells from/reduce off-target toxicity during chemotherapy, sensitize cells to chemotherapeutic agents, enhance survival of bone marrow transplants). In other embodiments, MANF/CDNF is administered with other compounds/agents antigens.

In some embodiments, the pharmaceutical composition is an immunogenic/vaccine composition that comprises both MANF and/or CDNF (as an adjuvant) and an antigen and may further comprise one or more components, as provided herein, that are selected from co-adjuvant and/or a recombinant expression construct, in combination with a pharmaceutically acceptable carrier, excipient or diluent.

The concentration of the active ingredient(s) in the formulations can vary depending upon a number of issues, including the dosage to be administered, and the route of administration.

For immunogenic compositions or vaccines comprising MANF and/or CDNF, including those comprising an antigen, about 0.001 μg/kg to about 100 mg/kg body weight will generally be administered, typically by the intradermal, subcutaneous, intramuscular, intranasally, or intravenous route, or by other routes.

In other embodiments, the dosage is about 0.001 μg/kg to about 1 mg/kg. In another embodiment, the dosage is about 0.001 to about 50 μg/kg. In another embodiment, the dosage is about 0.001 to about 15 μg/kg. In another embodiment, the amount of MANF and/or CDNF administered is about 0.01 μg/dose to about 5 mg/dose. In another embodiment, the amount of MANF and/or CDNF administered is about 0.1 μg/dose to about 1 mg/dose. In another embodiment, the amount of MANF and/or CDNF administered is about 0.1 μg/dose to about 100 μg/dose. In another embodiment, the MANF and/or CDNF administered is about 0.1 μg/dose to about 10 μg/dose.

It will be evident to those skilled in the art that the number and frequency of administration will be dependent upon the response of the host. “Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

The pharmaceutical compositions may be in any form that allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, and intranasal (e.g., as a spray) and also subcutaneous injections, intravenous, intramuscular, intrastemal, intracavernous, intrathecal, intrameatal, intraurethral injection or infusion techniques.

A MANF family protein can be administered prior to, concurrently, after therapy, e.g., immunotherapy for cancer, or administration of other agents/vaccine components.

Accordingly, in some embodiments, the present invention is useful for enhancing or eliciting, in a host, a patient or in cell culture, an immune response. As used herein, the term “subject” or “patient” refers to any warm-blooded animal, such as a human and domestic animals. A patient may be afflicted with an infectious disease, cancer, such as breast cancer, or an autoimmune disease, or may be normal (i.e., free of detectable disease and/or infection). A “cell culture” is any preparation containing immunocompetent cells or isolated cells of the immune system (including, but not limited to, T cells, macrophages, monocytes, B cells and dendritic cells).

Also contemplated in certain embodiments are kits comprising MANF and/or CDNF compositions, which can be provided in one or more containers. In one embodiment all components of the MANF and/or CDNF immunogenic/vaccine compositions and/or MANF and/or CDNF immunological adjuvant compositions are present together in a single container, but the invention embodiments are not intended to be so limited and also contemplate two or more containers in which, for example, a MANF and/or CDNF immunological adjuvant composition is separate from, and not in contact with, the antigen component. In some embodiments administration of the MANF and/or CDNF composition only can be performed beneficially, whilst in other cases such administration can beneficially be separated temporally and/or spatially (e.g., at a different anatomical site) from administration of the antigen, whilst in still other cases, administration to the subject is a composition comprising both antigen and MANF and/or CDNF, and optionally other herein described components as well.

### Specific Embodiments

For the purposes of clarity and a concise description, specific embodiments are provided below. These specific embodiments are meant to supplement, not replace, the preceding description. Further, the recitation of specific embodiments and definitions below does not exclude the combination of any of the embodiments below with the embodiments and description set forth above.

Embodiment 1. Provided is a method of enhancing an immunological response, the method comprising administering an immunogenic composition comprising an effective amount of a MANF family protein or fragment thereof and an antigen to a subject.

Embodiment 2. In the method of embodiment 1, the antigen can be immunologically cross-reactive with a pathogen.

Embodiment 3. In the method of embodiment 2, the pathogen can be a virus.

Embodiment 4. In the method of embodiment 3, the virus can be HIV-1, human herpes viruses, cytomegalovirus, Rotavirus, Epstein Barr virus, Varicella Zoster Virus, hepatitis B virus, hepatitis A virus, hepatitis C virus, hepatitis E virus, Respiratory Syncytial virus, parainfluenza virus, measles virus, mumps virus, human papilloma virus, Yellow Fever Virus, Dengue Virus, Tick-borne encephalitis virus, Japanese Encephalitis Virus, Ebola, or influenza virus.

Embodiment 5. In the method of embodiment 2, the pathogen can be a bacterium.

Embodiment 6. In the method of embodiment 5, the bacterium can be a Neisseria spp., Moraxella spp., Bordetella spp., Mycobacterium spp., Legionella spp., Escherichia spp., Vibrio spp., Shigella spp., Yersinia spp., Campylobacter spp., Salmonella spp., Listeria spp., Helicobacter spp., Pseudomonas spp., Staphylococcus spp., Enterococcus spp., Clostridium spp., Bacillus spp., Corynebacterium spp., Borrelia spp., Ehrlichia spp., Rickettsia spp., Chlamydia spp., Leptospira spp., or Treponema spp.

Embodiment 7. In the method of embodiment 5, the bacterium can be N. gonorrhea, N. meningitides, S. pyogenes, S. agalactiae, S. mutans, H. ducreyi, B. pertussis, B. parapertussis, B. bronchiseptica; M. tuberculosis, M. bovis, M. leprae, M. avium, M. paratuberculosis, M. smegmatis, L. pneumophila; enterotoxic E. coli, enterohemorragic E. coli, enteropathogenic E. coli, V. cholera, S. sonnei, S. dysenteriae, S. flexnerii, Y. enterocolitica, Y. pestis, Y. pseudotuberculosis, C. jejuni, C. coli, iS. typhi, S. paratyphi, S. choleraesuis, S. enteritidis, L. monocytogenes, H. pylori, P. aeruginosa, S. aureus, S. epidermidis, E. faecalis, E. faecium, C. tetani, C. botulinum, C. difficile, B. anthracis, C. diphtheria, B. burgdorferi, B. garinii, B. ajzelii, B. andersonii, B. hermsii, E. equi, the agent of the Human Granulocytic Ehrlichiosis, R. rickettsia, C. trachomatis, C. pneumoniae, C. psittaci, L. interrogans, T. pallidum, T. denticola, or T. hyodysenteriae.

Embodiment 8. In the method of embodiment 2, the pathogen can be a parasite.

Embodiment 9. In the method of embodiment 8, the parasite can be a Plasmodium spp., Toxoplasma spp., Entamoeba spp., Babesia spp., Trypanosoma spp., Giardia spp., Leishmania spp., Pneumocystis spp., or Trichomonas spp.

Embodiment 10. In the method of embodiment 8, the parasite can be P. falciparum, T. gondii, E. histolytica, B. microti, T. cruzi, G. lamblia, L. major, P. carinii, or T. vaginalis.

Embodiment 11. In the method of embodiment 8, the parasite can be a helminth.

Embodiment 12. In the method of embodiment 11, the helminth can be a nematode, trematode, or cestode.

Embodiment 13. In the method of embodiment 11, the helminth can be Enterobius vermicularis, Ascaris lumbricoides, Trichuris trichiura, Necator americanus, Ancylostoma duodenale, Wuchereria bancrofti, Brugia malayi, Onchocerca volvulus, Dracanculus medinensis, Trichinella spiralis, Strongyloides stercoralis, Schistosoma mansoni, Schistosoma haematobium, Schistosoma japonicum, Schistosoma mekongi, Opisthorchis sinensis, Paragonimus sp, Fasciola hepatica, Fasciola magna, Fasciola gigantica, Taenia saginata, or Taenia solium.

Embodiment 14. In the method of embodiment 2, the pathogen can be a yeast or other fungus.

Embodiment 15. In the method of embodiment 14, the yeast can be a Candida spp. or a Cryptococcus spp.

Embodiment 16. In the method of embodiment 14, the yeast can be C. albicans or C. neoformans.

Embodiment 17. In the method of embodiment 1, the antigen can be immunologically cross-reactive with a cancer cell.

Embodiment 18. In the method of embodiment 17, the cancer cell can be a prostate cancer cell, a breast cancer cell, a colorectal cancer cell, a lung cancer cell, a pancreatic cancer cell, a renal cancer cell, a melanoma cancer cell, an ovarian cancer cell, a B-cell malignancy cell, a leukemia cell, a lymphoma cell, a neuroblastoma cell, a glioblastoma cell, a skin cancer cell, or a combination thereof.

Embodiment 19. In the method of embodiment 1, the antigen can be a tumor rejection antigen.

Embodiment 20. In the method of embodiment 19, the tumor rejection antigen can be a patient specific antigen, a tumor specific antigen, or a tissue restricted antigen.

Embodiment 21. In the method of any one of embodiments 17-20, the subject can have breast cancer, colorectal cancer, lung cancer, pancreatic cancer, renal cancer, melanoma cancer, ovarian cancer, a B-cell malignancy, leukemia, lymphoma, a neuroblastoma, a glioblastoma, skin cancer, a liver cancer, a testicular cancer, an adrenal cancer, esophageal cancer, a sarcoma, a gastrointestinal cancer, a cervical cancer, a bone cancer or a combination thereof.

Embodiment 22. In the method of any one of embodiments 17-20, the subject can have a family history of breast cancer, colorectal cancer, lung cancer, pancreatic cancer, renal cancer, melanoma cancer, ovarian cancer, a B-cell malignancy, leukemia, lymphoma, a neuroblastoma, a glioblastoma, skin cancer, a liver cancer, a testicular cancer, an adrenal cancer, esophageal cancer, a sarcoma, a gastrointestinal cancer, a cervical cancer, a bone cancer or a combination thereof.

Embodiment 23. In the method of any one of embodiments 17-20, the subject can have an elevated risk of developing breast cancer, colorectal cancer, lung cancer, pancreatic cancer, renal cancer, melanoma cancer, ovarian cancer, a B-cell malignancy, leukemia, lymphoma, a neuroblastoma, a glioblastoma, skin cancer, a liver cancer, a testicular cancer, an adrenal cancer, esophageal cancer, a sarcoma, a gastrointestinal cancer, a cervical cancer, a bone cancer or a combination thereof.

Embodiment 24. Provided is a method of treating cancer comprising administering an immunologic composition comprising an effective amount of a MANF family protein or fragment thereof and a tumor rejection antigen to a subject in need thereof.

Embodiment 25. In the method of embodiment 24, the tumor rejection antigen can be a patient specific antigen.

Embodiment 26. In the method of embodiment 24, the tumor rejection antigen can be a tumor specific antigen.

Embodiment 27. In the method of embodiment 24, the tumor rejection antigen can be a tissue restricted antigen.

Embodiment 28. In the method of any one of embodiments 24-27, the subject in need thereof can have breast cancer, colorectal cancer, lung cancer, pancreatic cancer, renal cancer, melanoma cancer, ovarian cancer, a B-cell malignancy, leukemia, lymphoma, a neuroblastoma, a glioblastoma, skin cancer, a liver cancer, a testicular cancer, an adrenal cancer, esophageal cancer, a sarcoma, a gastrointestinal cancer, a cervical cancer, a bone cancer or a combination thereof.

Embodiment 29. In the method of any one of embodiments 24-28, the immunologic composition can further comprise an aluminum based salt, a squalene-oil-water emulsion, Bacillus Calmette-Guerin (BCG), or a combination thereof.

Embodiment 30. In the method of any one of embodiments 1-29, the MANF family protein or fragment thereof can be mesencephalic astrocyte derived neurotrophic factor (MANF) or a fragment thereof.

Embodiment 31. In the method of embodiment 30, the MANF or fragment thereof can comprise a peptide sequence that has at least about 80% identity with SEQ ID NO:3.

Embodiment 32. In the method of embodiment 30, the MANF or fragment thereof can comprise a peptide sequence that has at least about 90% identity with SEQ ID NO:3.

Embodiment 33. In the method of embodiment 30, the MANF or fragment thereof can comprise a peptide sequence that has at least about 95% identity with SEQ ID NO:3.

Embodiment 34. In the method of embodiment 30, the MANF or fragment thereof can comprise a peptide sequence that has at least about 80% identity with SEQ ID NO:3.

Embodiment 35. In the method of embodiment 30, the MANF or fragment thereof can comprise a peptide sequence that has at least about 90% identity with SEQ ID NO:3.

Embodiment 36. In the method of embodiment 30, the MANF or fragment thereof can comprise a peptide sequence that has at least about 95% identity with SEQ ID NO:3.

Embodiment 37. In the method of embodiment 30, the MANF or fragment thereof can consist of a peptide sequence that has 100% identity with SEQ ID NO:3.

Embodiment 38. In the method of any one of embodiments 30-37, the MANF or fragment thereof can have a length that is at least 80% the length of SEQ ID NO:3.

Embodiment 39. In the method of any one of embodiments 30-37, the MANF or fragment thereof can have a length that is 100% the length of SEQ ID NO:3.

Embodiment 40. In the method of embodiment 30, the MANF or fragment thereof can consist of a sequence listed in Table 3.

Embodiment 41. In the method of embodiment 40, MANF or fragment thereof can be cell permeable.

Embodiment 42. In the method of any one of embodiments 1-29, the MANF family protein or fragment thereof can be conserved dopaminergic neurotrophic factor (CDNF) or a fragment thereof.

Embodiment 43. In the method of embodiment 42, the CDNF or fragment thereof can comprise a peptide sequence that has at least about 80% identity with SEQ ID NO:6.

Embodiment 44. In the method of embodiment 42, the CDNF or fragment thereof can comprise a peptide sequence that has at least about 90% identity with SEQ ID NO:6.

Embodiment 45. In the method of embodiment 42, the CDNF or fragment thereof can comprise a peptide sequence that has at least about 95% identity with SEQ ID NO:6.

Embodiment 46. In the method of embodiment 42, the CDNF or fragment thereof can comprise a peptide sequence that has at least about 80% identity with SEQ ID NO:6.

Embodiment 47. In the method of embodiment 42, the CDNF or fragment thereof can comprise a peptide sequence that has at least about 90% identity with SEQ ID NO:6.

Embodiment 48. In the method of embodiment 42, the CDNF or fragment thereof can comprise a peptide sequence that has at least about 95% identity with SEQ ID NO:6.

Embodiment 49. In the method of embodiment 42, the CDNF or fragment thereof can consist of a peptide sequence that can be SEQ ID NO:6.

Embodiment 50. In the method of any one of embodiments 42-49, the CDNF or fragment thereof can have a length that is at least 80% the length of SEQ ID NO:6.

Embodiment 51. In the method of any one of embodiments 42-49, the CDNF or fragment thereof can have a length that is 100% the length of SEQ ID NO:6.

Embodiment 52. In the method of embodiment 42, the CDNF or fragment thereof can consist of a peptide sequence listed in Table 4.

Embodiment 53. In the method of embodiment 52, the CDNF or fragment thereof can be cell permeable.

Embodiment 54. In the method of any one of embodiments 1-53, the effective amount of the MANF family protein or fragment thereof can be from about 0.001 mg/kg to about 45 mg/kg.

Embodiment 55. In the method of any one of embodiments 1-53, the effective amount of the MANF family protein or fragment thereof can be about 0.1 mg/kg to about 45 mg/kg.

Embodiment 56. In the method of any one of embodiments 1-53, the effective amount of the MANF family protein or fragment thereof can be about 1 μg-500 μg.

Embodiment 57. In the method of any one of embodiments 1-53, the effective amount of the MANF family protein or fragment thereof can be about 5 μg-250 μg.

Embodiment 58. In the method of any one of embodiments 1-57, the immunologic composition can be administered by injection.

Embodiment 59. In the method of embodiment 58, the injection can be an intramuscular injection, a cubcutaneous injection, an intravenous injection, or a combination thereof.

Embodiment 60. In the method of any one of embodiments 1-57, the immunologic composition can be administered by intradermal administration.

Embodiment 61. In the method of any one of embodiments 1-57, the immunologic composition can be administered by intranasal administration.

Embodiment 62. In the method of any one of embodiments 1-61, further comprising administering one or more booster compositions that comprise the antigen or tumor rejection antigen without the MANF family protein.

Embodiment 63. In the method of any one of embodiments 1-61, further comprising administering one or more booster compositions that comprise the antigen without the MANF family protein over a period of weeks, months, or years.

Embodiment 64. In the method of any one of embodiments 1-61, the immunogenic composition can be administered to the subject two or more times.

Embodiment 65. In the method of any one of embodiments 1-61, the immunogenic composition can be administered to the subject two or more times over a period of weeks, months, or years.

Embodiment 66. Provided is a method of treating cancer comprising administering to a subject in need thereof an effective amount of a MANF family protein or fragment thereof and genetically engineered T cells expressing a chimeric antigen receptor.

Embodiment 67. In the method of embodiment 66, the chimeric antigen receptor can comprise an antigen recognition region and an endodomain.

Embodiment 68. In the method of embodiment 67, the target antigen of the antigen recognition region can be α-Folate receptor, CALX, CD19, CD20, CD22, CD30, CD33, CD44v7/8, CEA, EGP-2, EGP-40, erb-B2, erb-B 2,3,4, FBP, Fetal acetylcholine receptor, GD2, GD3, Her2/neu, IL-13R-a2, KDR, k-light chain, LeY, L1 cell adhesion molecule, MAGE-A1, Mesothelin, Murine CMV infected cells, MUC1, NKG2D ligands, Oncofetal antigen (h5T4), PSCA, PSMA, TAA targeted by mAb IgE, TAG-72, or VEGF-R2.

Embodiment 69. In the method of any one of embodiments 67-68, the endodomain can comprise ScFv-FcεRIγCAIX, ScFv-FcεRIγ, ScFv-CD3ζ (EBV), ScFv-CD3ζ, ScFv-CD28-CD3ζ, CD3ζ(EBV), ScFv-CD28-CD3ζ, CD3ζ, ScFv-CD3ζ, ScFv-41BB-CD3ζ, ScFv-41BB-CD3ζ, ScFv-CD3ζ (Influenza MP-1), ScFv-CD3ζ (VZV), ScFv-CD4-CD3ζ, CD3 ζ/CD137/CD28, ScFv-CD28-41BB-CD3ζ, ScFv-CD8-CD3ζ, ScFv-FcεRIγ, CD28/4-1BB-CD3ζ, ScFv-CD28-CD3ζ (Influenza), ScFv-CD28mut-CD3ζ, Heregulin-CD3ζ, ScFv-FcεRIγ (alloantigen), ScFv-CD28, ScFv-CD28-OX40-CD3ζ, ScFv-CD3ζ, IL-13-CD28-4-1BB-CD3ζ, IL-13-CD3ζ, ScFv-CD28-CD3ζ, ScFV-CD4-FcεRIγ, ScFV-CD28-FcεRIγ, Ly49H-CD3ζ, NKG2D-CD3ζ, ScFV-CD3ζ (vaccination), ScFv-b2c-CD3ζ, or FceRI-CD28-CD3ζ (+a-TAA IgE mAb).

Embodiment 70. In the method of any one of embodiments 66-69, the genetically engineered T cells were grown in the presence of the MANF family protein or fragment thereof.

Embodiment 71. In the method of any one of embodiments 66-69, the genetically engineered T cells were primed with the MANF family protein or fragment thereof.

Embodiment 72. In the method of any one of embodiments 66-69, the genetically engineered T cells were primed for about 10 seconds to about 1 hour with the MANF family protein or fragment thereof.

Embodiment 73. In the method of any one of embodiments 66-72, the effective amount of the MANF family protein or fragment thereof can be from about 0.001 mg/kg to about 45 mg/kg.

Embodiment 74. In the method of any one of embodiments 66-72, the effective amount of the MANF family protein or fragment thereof can be about 0.1 mg/kg to about 45 mg/kg.

Embodiment 75. In the method of any one of embodiments 66-72, the effective amount of the MANF family protein or fragment thereof can be about 1 μg-500 μg.

Embodiment 76. In the method of any one of embodiments 66-72, the effective amount of the MANF family protein can be about 5 μg-250 μg.

Embodiment 77. In the method of any one of embodiments 66-76, the effective amount of the MANF family protein or fragment thereof can be sufficient to increase cell viability of the genetically engineered T cells.

Embodiment 78. In the method of any one of embodiments 66-77, the MANF family protein or fragment thereof can be mesencephalic astrocyte derived neurotrophic factor (MANF) or a fragment thereof.

Embodiment 79. In the method of embodiment 78, the MANF or fragment thereof can comprise a peptide sequence that has at least about 80% identity with SEQ ID NO:3.

Embodiment 80. In the method of embodiment 78, the MANF or fragment thereof can comprise a peptide sequence that has at least about 90% identity with SEQ ID NO:3.

Embodiment 81. In the method of embodiment 78, the MANF or fragment thereof can comprise a peptide sequence that has at least about 95% identity with SEQ ID NO:3.

Embodiment 82. In the method of embodiment 78, the MANF or fragment thereof can comprise a peptide sequence that has at least about 80% identity with SEQ ID NO:3.

Embodiment 83. In the method of embodiment 78, the MANF or fragment thereof can comprise a peptide sequence that has at least about 90% identity with SEQ ID NO:3.

Embodiment 84. In the method of embodiment 78, the MANF or fragment thereof can comprise a peptide sequence that has at least about 95% identity with SEQ ID NO:3.

Embodiment 85. In the method of embodiment 78, the MANF or fragment thereof can consist of a peptide sequence that has 100% identity with SEQ ID NO:3.

Embodiment 86. In the method of any one of embodiments 78-85, the MANF or fragment thereof can have a length that is at least 80% the length of SEQ ID NO:3.

Embodiment 87. In the method of any one of embodiments 78-85, the MANF or fragment thereof can have a length that is 100% the length of SEQ ID NO:3.

Embodiment 88. In the method of embodiment 78, the MANF or fragment thereof can consist of a sequence listed in Table 3.

Embodiment 89. In the method of embodiment 88, MANF or fragment thereof can be cell permeable.

Embodiment 90. In the method of any one of embodiments 66-77, the MANF family protein or fragment thereof can be conserved dopaminergic neurotrophic factor (CDNF) or a fragment thereof.

Embodiment 91. In the method of embodiment 90, the CDNF or fragment thereof can comprise a peptide sequence that has at least about 80% identity with SEQ ID NO:6.

Embodiment 92. In the method of embodiment 90, the CDNF or fragment thereof can comprise a peptide sequence that has at least about 90% identity with SEQ ID NO:6.

Embodiment 93. In the method of embodiment 90, the CDNF or fragment thereof can comprise a peptide sequence that has at least about 95% identity with SEQ ID NO:6.

Embodiment 94. In the method of embodiment 90, the CDNF or fragment thereof can comprise a peptide sequence that has at least about 80% identity with SEQ ID NO:6.

Embodiment 95. In the method of embodiment 90, the CDNF or fragment thereof can comprise a peptide sequence that has at least about 90% identity with SEQ ID NO:6.

Embodiment 96. In the method of embodiment 90, the CDNF or fragment thereof can comprise a peptide sequence that has at least about 95% identity with SEQ ID NO:6.

Embodiment 97. In the method of embodiment 90, the CDNF or fragment thereof can consist of a peptide sequence that can be SEQ ID NO:6.

Embodiment 98. In the method of any one of embodiments 90-97, the CDNF or fragment thereof can have a length that is at least 80% the length of SEQ ID NO:6.

Embodiment 99. In the method of any one of embodiments 90-97, the CDNF or fragment thereof can have a length that is 100% the length of SEQ ID NO:6.

Embodiment 100. In the method of embodiment 90, the CDNF or fragment thereof can consist of a peptide sequence listed in Table 4.

Embodiment 101. In the method of embodiment 100, the CDNF or fragment thereof can be cell permeable.

Embodiment 102. Provided is a method of treating cancer comprising administering an effective amount of a MANF family protein or fragment thereof and a bispecific monoclonal antibody to a subject in need thereof.

Embodiment 103. In the method of embodiment 102, the bispecific antibody can comprise a tumor antigen epitope and a cytotoxic cell epitope.

Embodiment 104. In the method of embodiment 103, the tumor antigen epitope binds to a tumor antigen on a prostate cancer cell, a breast cancer cell, a colorectal cancer cell, a lung cancer cell, a pancreatic cancer cell, a renal cancer cell, a melanoma cancer cell, an ovarian cancer cell, a B-cell malignancy cell, a leukemia cell, a lymphoma cell, a neuroblastoma cell, a glioblastoma cell, a skin cancer cell, a liver cancer cell, a testicular cancer cell, an adrenal cancer cell, esophageal cancer cell, a sarcoma cell, a gastrointestinal cancer cell, a cervical cancer cell, a bone cancer cell or a combination thereof.

Embodiment 105. In the method of any one of embodiments 103-104, the cytotoxic cell epitope binds to a cytotoxic cell antigen on a T lymphocyte, a macrophage, a natural killer cell, a dendritic cell, or a combination thereof.

Embodiment 106. In the method of any one of embodiments 102-105, the effective amount of the MANF family protein or fragment thereof can be from about 0.001 mg/kg to about 45 mg/kg.

Embodiment 107. In the method of any one of embodiments 102-105, the effective amount of the MANF family protein or fragment thereof can be about 0.1 mg/kg to about 45 mg/kg.

Embodiment 108. In the method of any one of embodiments 102-105, the effective amount of the MANF family protein or fragment thereof can be about 1 μg-500 μg.

Embodiment 109. In the method of any one of embodiments 102-105, the effective amount of the MANF family protein can be about 5 μg-250 μg.

Embodiment 110. In the method of any one of embodiments 102-105, the effective amount of the MANF family protein or fragment thereof can be sufficient to enhance the survivability of endogenous cytotoxic cells.

Embodiment 111. In the method of any one of embodiments 102-105, the effective amount of the MANF family protein or fragment thereof can be sufficient enhance an immune response against tumor cells.

Embodiment 112. In the method of any one of embodiments 102-110, the MANF family protein or fragment thereof can be mesencephalic astrocyte derived neurotrophic factor (MANF) or a fragment thereof.

Embodiment 113. In the method of embodiment 111, the MANF or fragment thereof can comprise a peptide sequence that has at least about 80% identity with SEQ ID NO:3.

Embodiment 114. In the method of embodiment 111, the MANF or fragment thereof can comprise a peptide sequence that has at least about 90% identity with SEQ ID NO:3.

Embodiment 115. In the method of embodiment 111, the MANF or fragment thereof can comprise a peptide sequence that has at least about 95% identity with SEQ ID NO:3.

Embodiment 116. In the method of embodiment 111, the MANF or fragment thereof can comprise a peptide sequence that has at least about 80% identity with SEQ ID NO:3.

Embodiment 117. In the method of embodiment 111, the MANF or fragment thereof can comprise a peptide sequence that has at least about 90% identity with SEQ ID NO:3.

Embodiment 118. In the method of embodiment 111, the MANF or fragment thereof can comprise a peptide sequence that has at least about 95% identity with SEQ ID NO:3.

Embodiment 119. In the method of embodiment 111, the MANF or fragment thereof can consist of a peptide sequence that has 100% identity with SEQ ID NO:3.

Embodiment 120. In the method of any one of embodiments 111-118, the MANF or fragment thereof can have a length that is at least 80% the length of SEQ ID NO:3.

Embodiment 121. In the method of any one of embodiments 111-118, the MANF or fragment thereof can have a length that is 100% the length of SEQ ID NO:3.

Embodiment 122. In the method of embodiment 111, the MANF or fragment thereof can consist of a sequence listed in Table 3.

Embodiment 123. In the method of embodiment 121, MANF or fragment thereof can be cell permeable.

Embodiment 124. In the method of any one of embodiments 102-110, the MANF family protein or fragment thereof can be conserved dopaminergic neurotrophic factor (CDNF) or a fragment thereof.

Embodiment 125. In the method of embodiment 123, the CDNF or fragment thereof can comprise a peptide sequence that has at least about 80% identity with SEQ ID NO:6.

Embodiment 126. In the method of embodiment 123, the CDNF or fragment thereof can comprise a peptide sequence that has at least about 90% identity with SEQ ID NO:6.

Embodiment 127. In the method of embodiment 123, the CDNF or fragment thereof can comprise a peptide sequence that has at least about 95% identity with SEQ ID NO:6.

Embodiment 128. In the method of embodiment 123, the CDNF or fragment thereof can comprise a peptide sequence that has at least about 80% identity with SEQ ID NO:6.

Embodiment 129. In the method of embodiment 123, the CDNF or fragment thereof can comprise a peptide sequence that has at least about 90% identity with SEQ ID NO:6.

Embodiment 130. In the method of embodiment 123, the CDNF or fragment thereof can comprise a peptide sequence that has at least about 95% identity with SEQ ID NO:6.

Embodiment 131. In the method of embodiment 123, the CDNF or fragment thereof can consist of a peptide sequence that can be SEQ ID NO:6.

Embodiment 132. In the method of any one of embodiments 123-130, the CDNF or fragment thereof can have a length that is at least 80% the length of SEQ ID NO:6.

Embodiment 133. In the method of any one of embodiments 123-130, the CDNF or fragment thereof can have a length that is 100% the length of SEQ ID NO:6.

Embodiment 134. In the method of embodiment 123, the CDNF or fragment thereof can consist of a peptide sequence listed in Table 4.

Embodiment 135. In the method of embodiment 133, the CDNF or fragment thereof can be cell permeable.

Embodiment 136. In the method of any one of embodiments 102-135, the MANF family protein or fragment thereof can be administered with the bispecific monoclonal antibody.

Embodiment 137. In the method of any one of embodiments 102-135, the MANF family protein or fragment thereof can be administered before the bispecific monoclonal antibody.

Embodiment 138. In the method of any one of embodiments 102-135, the MANF family protein or fragment thereof can be administered after the bispecific monoclonal antibody.

Embodiment 139. In the method of any one of embodiments 102-138, administration can be by injection.

Embodiment 140. Provided is a method to increase survival of a bone marrow transplant comprising administering an effective amount of a MANF family protein or fragment thereof to a subject undergoing a bone marrow transplant, so as to increase the survival of the bone marrow transplant as compared to a bone marrow transplant in the absence of the MANF family protein.

Embodiment 141. In the method of embodiment 140, the MANF family protein or fragment thereof can be administered to the subject prior to the bone marrow transplant.

Embodiment 142. In the method of embodiment 140, the MANF family protein or fragment thereof can be administered to the subject concurrently with the bone marrow transplant.

Embodiment 143. In the method of embodiment 140, the MANF family protein or fragment thereof can be administered to the subject following the bone marrow transplant.

Embodiment 144. In the method of any one of embodiments 141-143, the MANF family protein or fragment thereof can be administered to the site of the bone marrow transplant in the subject.

Embodiment 145. In the method of any one of embodiments 141-144, further comprising contacting bone marrow cells of the bone marrow transplant with the MANF family protein or fragment thereof prior to the bone marrow transplant.

Embodiment 146. Provided is a method to increase survival of a bone marrow transplant comprising contacting allogenic or autologous bone marrow cells ex vivo with an effective amount of a MANF family protein or fragment thereof thereby increasing the survival of the bone marrow transplant as compared to a bone marrow transplant in the absence of the MANF family protein.

Embodiment 147. Provided is a method of treating at least one condition associated with chemotherapy or radiation therapy comprising administering to a subject in need thereof an effective amount of a MANF family protein or fragment thereof. Optionally, the condition associated with chemotherapy or radiation therapy can exclude ototoxicity.

Embodiment 148. In the method of embodiment 147, the MANF family protein or fragment thereof can be administered to healthy tissue surrounding a tumor.

Embodiment 149. In the method of any one of embodiments 147-MS, the MANF family protein or fragment thereof can be administered prior to the chemotherapy or radiation therapy.

Embodiment 150. In the method of any one of embodiments MO-149, the effective amount of the MANF family protein or fragment thereof can be from about 0.001 mg/kg to about 45 mg/kg.

Embodiment 151. In the method of any one of embodiments 140-149, the effective amount of the MANF family protein or fragment thereof can be about 0.1 mg/kg to about 45 mg/kg.

Embodiment 152. In the method of any one of embodiments MO-149, the effective amount of the MANF family protein or fragment thereof can be about 1 μg-500 μg.

Embodiment 153. In the method of any one of embodiments MO-149, the effective amount of the MANF family protein can be about 5 μg-250 μg.

Embodiment 154. In the method of any one of embodiments MO-153, the MANF family protein or fragment thereof can be mesencephalic astrocyte derived neurotrophic factor (MANF) or a fragment thereof.

Embodiment 155. In the method of embodiment 154, the MANF or fragment thereof can comprise a peptide sequence that has at least about 80% identity with SEQ ID NO:3.

Embodiment 156. In the method of embodiment 154, the MANF or fragment thereof can comprise a peptide sequence that has at least about 90% identity with SEQ ID NO:3.

Embodiment 157. In the method of embodiment 154, the MANF or fragment thereof can comprise a peptide sequence that has at least about 95% identity with SEQ ID NO:3.

Embodiment 158. In the method of embodiment 154, the MANF or fragment thereof can comprise a peptide sequence that has at least about 80% identity with SEQ ID NO:3.

Embodiment 159. In the method of embodiment 154, the MANF or fragment thereof can comprise a peptide sequence that has at least about 90% identity with SEQ ID NO:3.

Embodiment 160. In the method of embodiment 154, the MANF or fragment thereof can comprise a peptide sequence that has at least about 95% identity with SEQ ID NO:3.

Embodiment 161. In the method of embodiment 154, the MANF or fragment thereof can consist of a peptide sequence that has 100% identity with SEQ ID NO:3.

Embodiment 162. In the method of any one of embodiments 154-161, the MANF or fragment thereof can have a length that is at least 80% the length of SEQ ID NO:3.

Embodiment 163. In the method of any one of embodiments 154-lb 1, the MANF or fragment thereof can have a length that is 100% the length of SEQ ID NO:3.

Embodiment 164. In the method of embodiment 154, the MANF or fragment thereof can consist of a sequence listed in Table 3.

Embodiment 165. In the method of embodiment 164, MANF or fragment thereof can be cell permeable.

Embodiment 166. In the method of any one of embodiments MO-153, the MANF family protein or fragment thereof can be conserved dopaminergic neurotrophic factor (CDNF) or a fragment thereof.

Embodiment 167. In the method of embodiment 166, the CDNF or fragment thereof can comprise a peptide sequence that has at least about 80% identity with SEQ ID NO:6.

Embodiment 168. In the method of embodiment 166, the CDNF or fragment thereof can comprise a peptide sequence that has at least about 90% identity with SEQ ID NO:6.

Embodiment 169. In the method of embodiment 166, the CDNF or fragment thereof can comprise a peptide sequence that has at least about 95% identity with SEQ ID NO:6.

Embodiment 170. In the method of embodiment 166, the CDNF or fragment thereof can comprise a peptide sequence that has at least about 80% identity with SEQ ID NO:6.

Embodiment 171. In the method of embodiment 166, the CDNF or fragment thereof can comprise a peptide sequence that has at least about 90% identity with SEQ ID NO:6.

Embodiment 172. In the method of embodiment 166, the CDNF or fragment thereof can comprise a peptide sequence that has at least about 95% identity with SEQ ID NO:6.

Embodiment 173. In the method of embodiment 166, the CDNF or fragment thereof can consist of a peptide sequence that can be SEQ ID NO:6.

Embodiment 174. In the method of any one of embodiments 166-173, the CDNF or fragment thereof can have a length that is at least 80% the length of SEQ ID NO:6.

Embodiment 175. In the method of any one of embodiments 166-173, the CDNF or fragment thereof can have a length that is 100% the length of SEQ ID NO:6.

Embodiment 176. In the method of embodiment 166, the CDNF or fragment thereof can consist of a peptide sequence listed in Table 4.

Embodiment 177. In the method of embodiment 176, the CDNF or fragment thereof can be cell permeable.

Embodiment 178. Provided is a method to increase survival or reduce tumor burden/size in a subject that has cancer comprising administering to the a MANF family protein antagonist, administration of the MANF family protein antagonist to the subject increases survival of the subject or reduces tumor burden/size in the subject, the MANF family protein antagonist reduces the level or activity of a MANF family protein.

Embodiment 179. Provided is a method to sensitize cells to at least one chemotherapeutic agent comprising administering a MANF family protein antagonist to a subject who can be or will be undergoing chemotherapy, so as to sensitize cells to the chemotherapy as compared to chemotherapy in the absence of the MANF family protein antagonist.

Embodiment 180. Provided is a method to target self-reactive immune cells comprising administering a MANF family protein antagonist to a subject in need thereof, the MANF family protein antagonist reduces the level and/or activity of at least one MANF family protein, so as to treat an auto-immune disorder.

Embodiment 181. Provided is a method to treat an immune-mediated inflammatory disease (IMID) comprising administering a MANF family protein antagonist to a subject in need thereof, the MANF family protein antagonist reduces the level and/or activity of at least one MANF family protein, so as to treat an auto-immune disorder.

Embodiment 182. In the method of embodiment 180 or 181, the subject has multiple sclerosis, irritable bowel syndrome, lupus, myasthenia gravis, rheumatoid arthritis, Hashimoto's thyroiditis, Grave's disease, autoimmune hepatitis, Alopecia, Addison's disease, Psoriasis, autoimmune pancreatitis, Celiac disease, pernicious anemia, Still's disease, juvenile arthritis, Felty syndrome, relapsing polychondritis, Guillain-Barré syndrome, vasculitis, or Crohn's disease.

Embodiment 183. In the method of any one of embodiments 178-182, the MANF family protein antagonist can be an anti-MANF antibody.

Embodiment 184. In the method of any one of embodiments 178-182, the MANF family protein antagonist can be an anti-CDNF antibody.

Embodiment 185. In the method of any one of embodiments 178-182, the MANF family protein antagonist can be an siRNA directed against a MANF gene.

Embodiment 186. In the method of any one of embodiments 178-182, the MANF family protein antagonist can be an siRNA directed against a CDNF gene.

The following non-limiting Examples illustrate some of the experimental work involved in developing the invention.

### EXAMPLES

### Example 1: MANF in B-Cell Responses

To obtain a general idea about MANF expression in the immune system, the publically available Human Protein Atlas (http://www.proteinatlas.org) was surveyed.

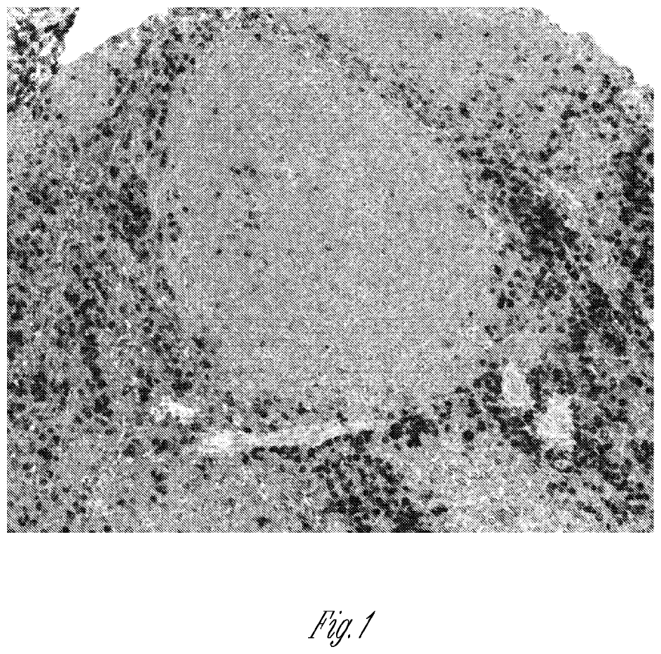

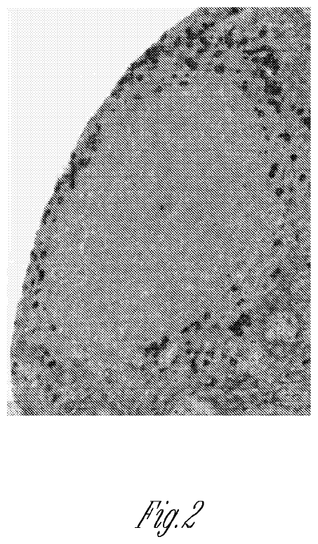

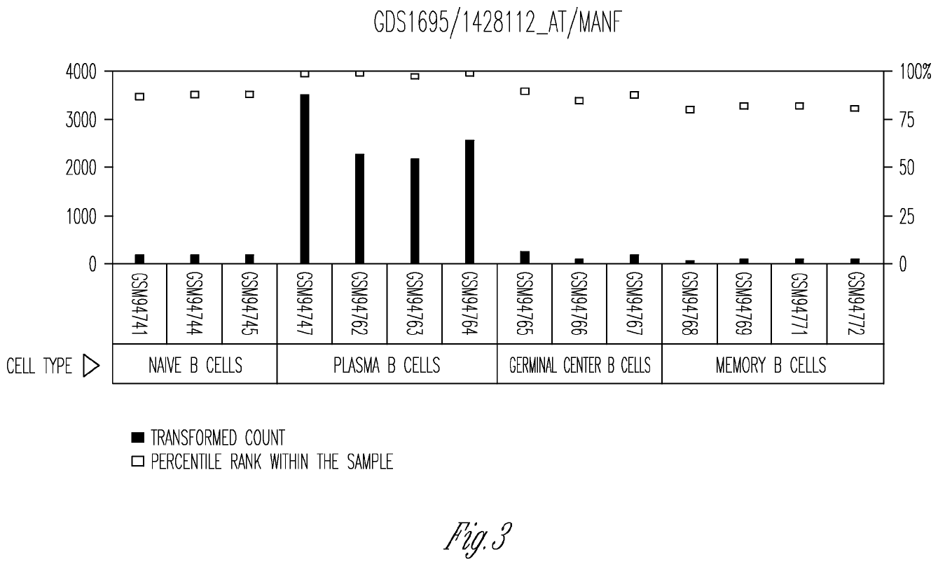

MANF is expressed in T and B cells within the immune system as shown by the immunohistochemical staining of the non-germinal cells within the lymph nodes of both the spleen (FIG. 1) and tonsil (FIG. 2). This is an area of both T and B cell accumulation and interaction.

Next, the National Center for Biotechnology Information (NCBI) Gene Expression Omnibus (GEO) repository (available at: www.ncbi.nlm.nih.gov/geoprofiles/) was reviewed for MANF related data from microarray studies that have been deposited in this site.

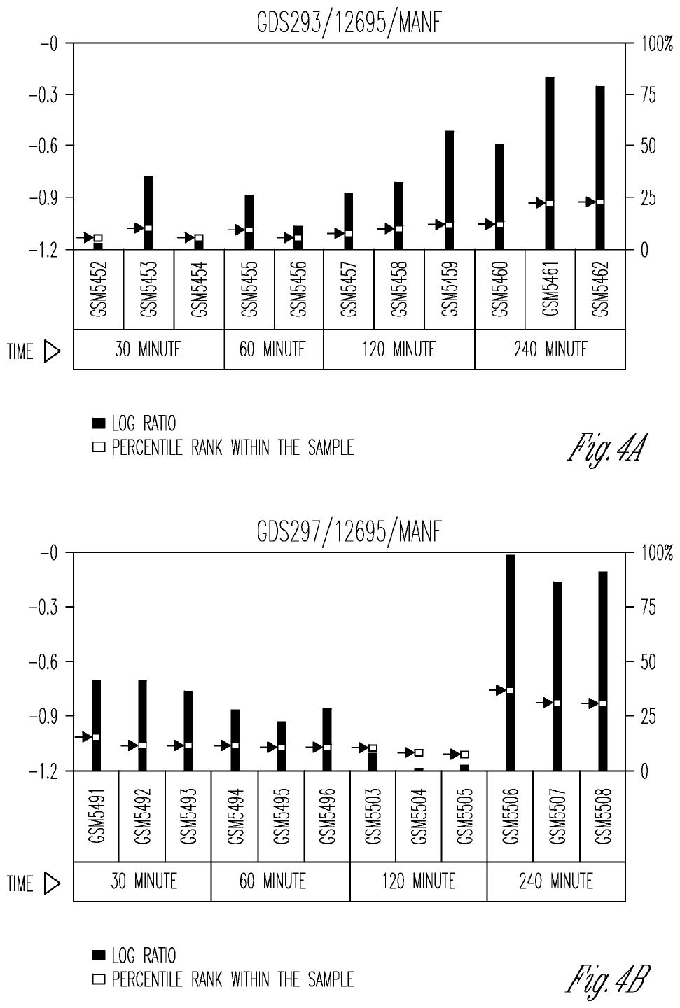

Within B cell populations, MANF is dramatically induced in response to immunization with the T-dependent antigen, NP-CGG, in plasma B cells relative to naïve B cells, germinal center B cells or memory B cells (FIG. 3). These results indicate that memory B cells may share a transcriptional program with memory T cells (mTCs) and long-term hermatopoietic stem cells (Lt-HSCs). These data also indicate that MANF expression may be predominantly observed in plasma B cells relative to naïve B cells, germinal B cells, or memory B cells.

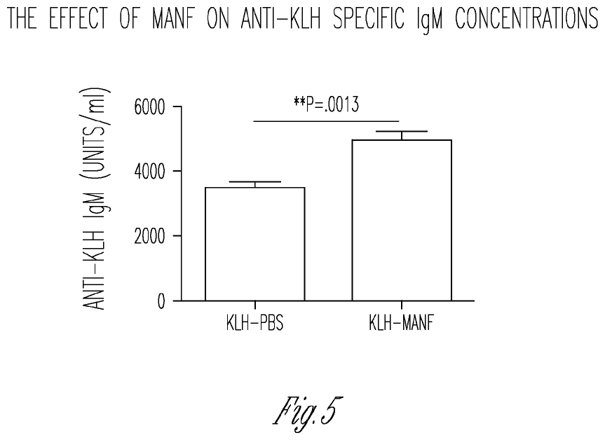

Studies with B cells from Mus musculus indicate that MANF is dynamically regulated during B cell activation. As shown in microarray expression data from ligand screens using male C57BL/6 B cells, B cell activation with either B-cell activating factor (FIG. 4A) or 65 nM CD40 (FIG. 4B) leads to a dramatic, but transient, repression of MANF expression in B cells (FIGS. 4A&B). This indicates that MANF expression may be suppressed when B cells first initiate mitosis following activation.

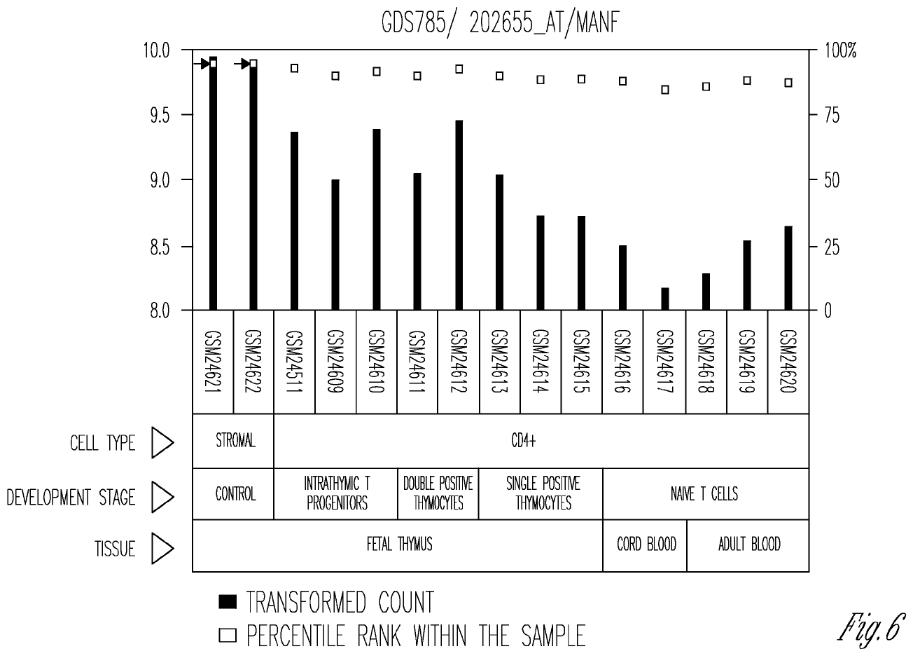

Based on the expression in the non-germinal cells within the lymph nodes it was speculated that MANF might enhance antibody production and provide for faster and/or higher titers of antibodies following exposure to a novel antigen. To test this hypothesis, 8 week old male mice were injected intraperitoneally with 100 μg of sterile TNP-KLH (2,4,6, Trinitrophenyl hapten conjugated Keyhole Limpet Hemocyanin) with 5% alum in the presence of 200 μL of 1 μg/μL MANF or an equal volume of PBS. Blood was sampled via the submandibular vein seven days later and the KLH-specific IgM analyzed using an enzyme linked immunoassays (ELISA) from Life Diagnostics, Inc. The concentration was calculated from a standard curve. A Student's t-test was used to establish statistical significance (p=0.0013). The presence of MANF significantly increased the levels of anti-KLH IgM antibodies (FIG. 5).

### Example 2: MANF in T Cell Responses

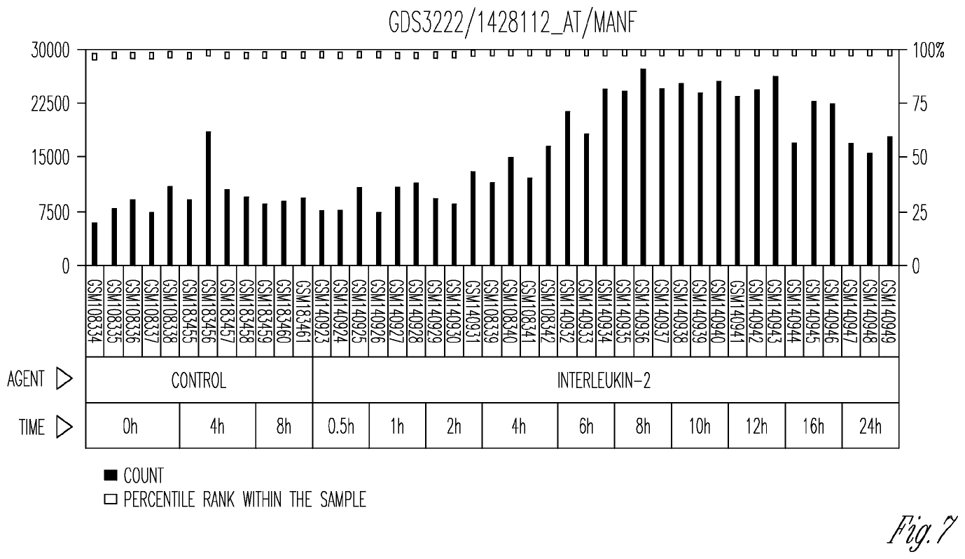

To extend our analysis to T cell responses, data from the Geo Database was examined. It was found that CD4+ T cells express MANF at all developmental stages examined, including intrathymic T progenitors, double positive thymocytes, single positive thymocytes, naïve T cells from cord blood, and naïve T cells from adult blood (FIG. 6).

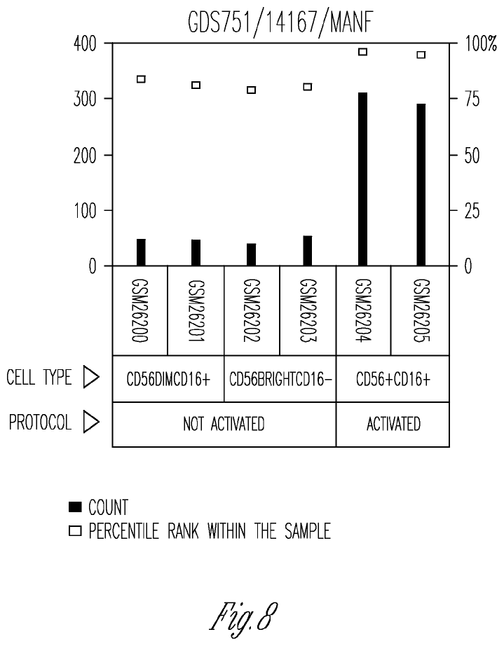

MANF is also expressed in a mouse cytotoxic T cell line (CTLL-2), and MANF expression levels become elevated when the cells are activated in response to interleukin-2 (IL-2) (FIG. 7). IL-2 regulates T cell proliferation and differentiation. The CTLL-2 data provides insight into the expression patterns of IL-2 responsive genes.

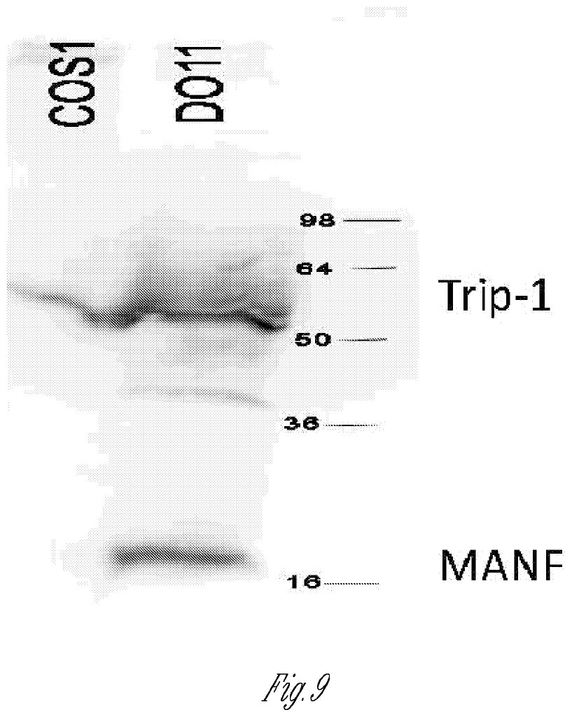

MANF is also expressed in human Natural Killer (NK) T cells (FIG. 8). The expression of MANF is higher in activated NK cells than in NK cells that were not activated.

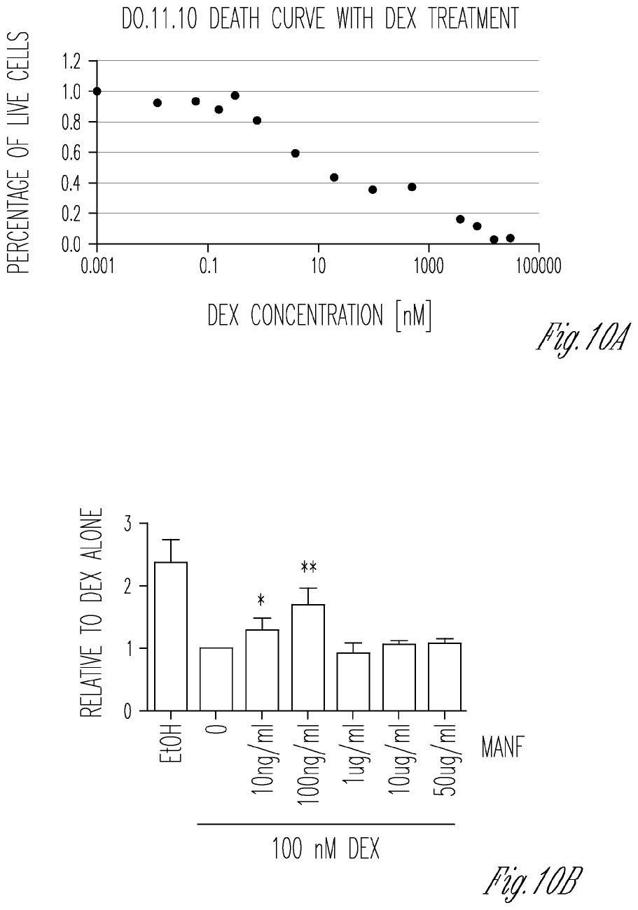

Because it was found that MANF is expressed in T cells as well as B cells, the functional roles of MANF in T cells was explored using the well-studied T cell hybrid DO 11.10 cells, which expresses MANF endogenously (FIG. 9). Western blot analysis demonstrates that DO 11.10 expresses MANF protein. COS-1 cells, which are derived from the kidney, were used as a control. The blot was re-probed with antibodies to the 26S proteasome subunit Trip-1, which was used as a loading control to demonstrate that both lanes contained equivalent amounts of cellular protein. DO 11.10 cells, but not COS-1 cells express endogenous MANF.

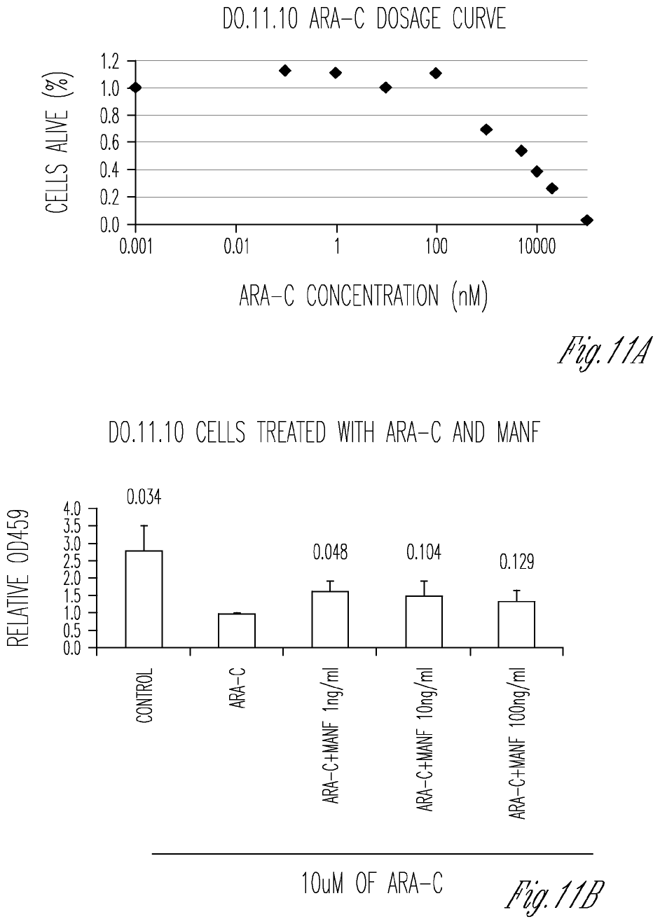

Because MANF is expressed in T cells and is induced when they become activated, experiments were performed to determine whether MANF confers protection to these cells. A dose-response curve was generated with the glucocorticoid dexamethasone to determine the dose that results in ˜50% cell death (24 nM) (FIG. 10A). DO 11.10 cells were then pretreated for 24 hours with 0, 10 ng/mL, 100 ng/mL, 1 μg/mL, 10 μg/mL, or 50 μg/mL MANF before exposure to 100 nM dexamethasone to investigate a protective effect for MANF. Exogenous MANF conferred statistically significant, does-dependent protection against dexamethasone induced cell death at both 10 ng/mL (p=0.003) and 100 ng/mL (p=0.019) (FIG. 10B). No statistically significant effects were seen at higher doses, which is similar to what has been observed with neuronal cultures. These are key functional data.

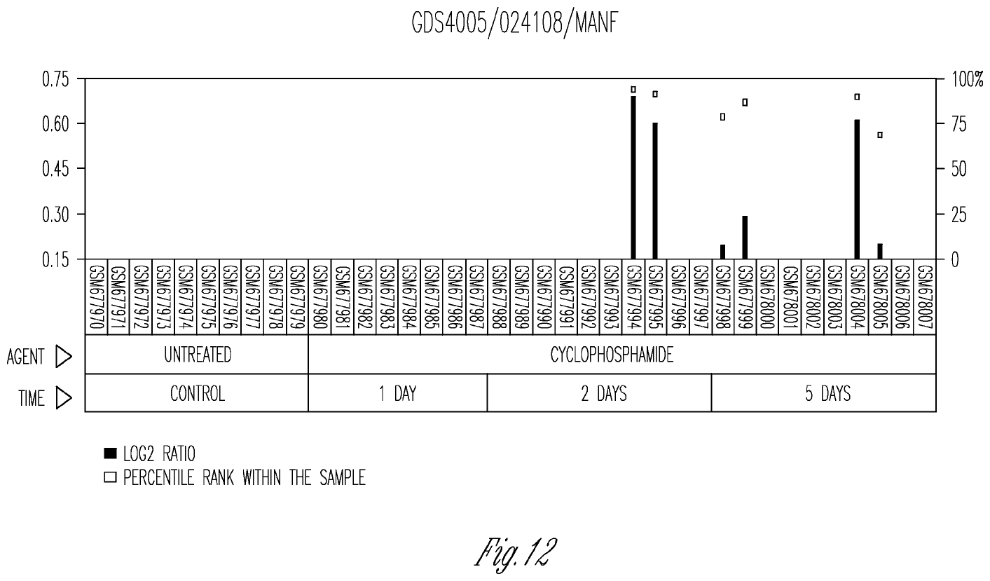

Similar results were obtained when DO 11.10 cells were challenged with cytosine arabinoside (Ara-C) a chemotherapeutic drug that is used to treat leukemias and lymphomas (FIGS. 11A&B). A dose-response curve was generated with the Ara-C to determine the dose that results in 50% cell death (FIG. 11A). The LD50, based on these data, was ˜5 μM. D010.10 cells were then pretreated for 24 hours with 0, 1 ng/mL, 10 ng/mL, or 100 ng/mL MANF before exposure to 10 μM Ara-C. MANF conferred dose-dependent protection against Ara-C at all concentrations tested, with the statistical significance indicated in FIG. 11B.

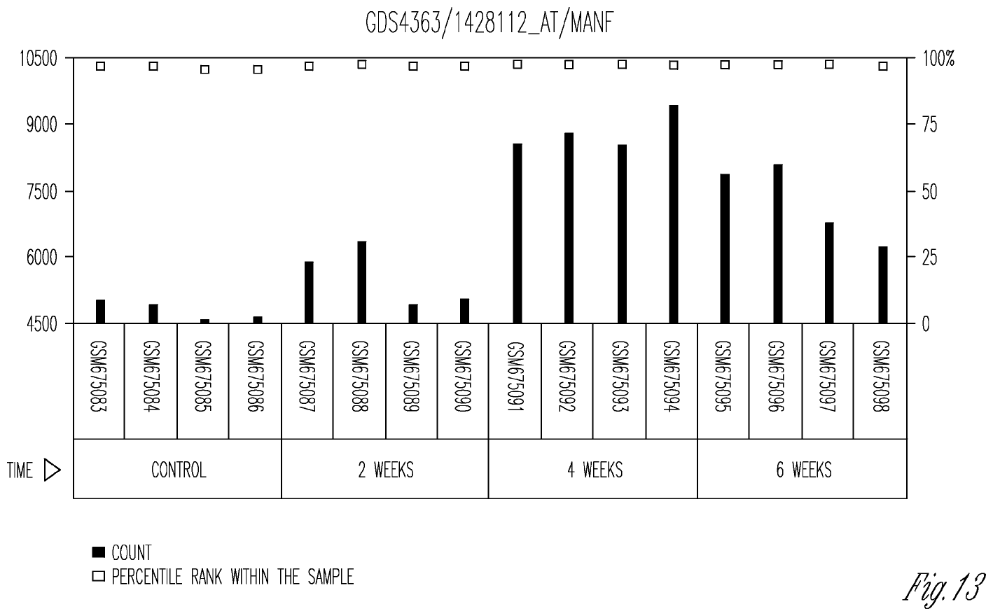

Because MANF confers some protection against chemotherapeutics, data from the GEO database was investigated to determine if MANF expression is induced by chemotherapeutic drugs. The data indicate that MANF is highly induced in mouse splenocytes that are treated with the toxic chemotherapeutic drug cyclophosphamide (CTX) (FIG. 12).

These data suggest that targeting MANF expression could impact on the efficacy of chemotherapeutic drug treatment. For example, ectopic MANF might protect non-cancerous cells from chemotherapy. Alternatively, focal administration of a MANF inhibitor agents (e.g., siRNA or anti-MANF antibody) might sensitize tumors and enhance tumor cell death.

### Example 3: MANF in CD4CD45 RBhigh T Cell Transfer Colitis

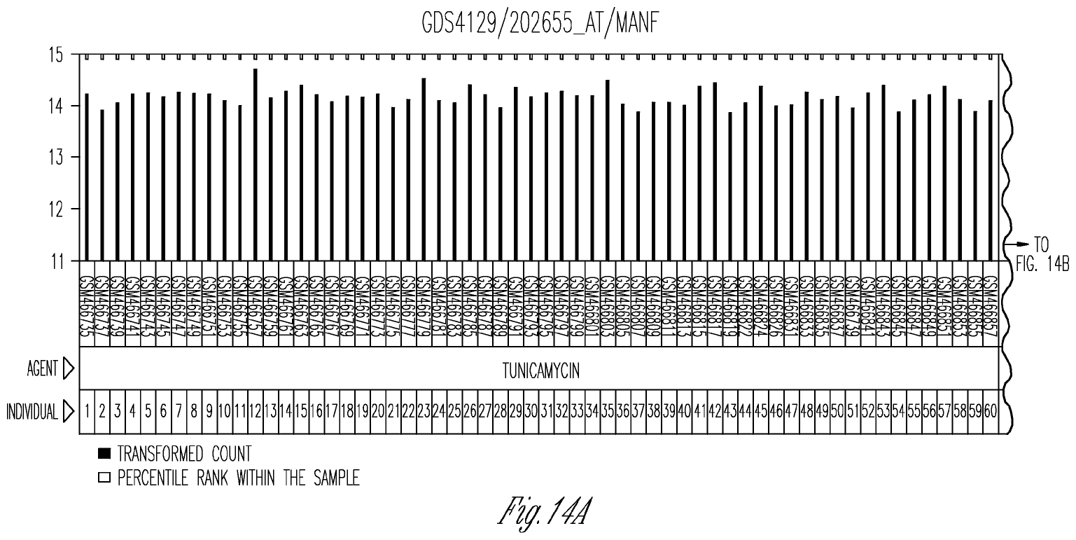

Data from the National Center for Biotechnology Information (NCBI) Gene Expression Omnibus (GEO) repository (available at: www.ncbi.nlm.nih.gov/geoprofiles/) was analyzed to determine MANF expression in colon tissue in an animal model of inflammatory bowel disease. MANF expression increases in colon tissue in the weeks following transfer of CD4+CD45RBhigh T cells from healthy wild type C57BL/6 males to RAG-1−/−C57BL/6 males in order to induce colitis (FIG. 13). These data provide insight into the molecular basis of colitis development.

### Example 4: MANF Expression in B Cells Undergoing ER Stress and the Unfolded Protein Response

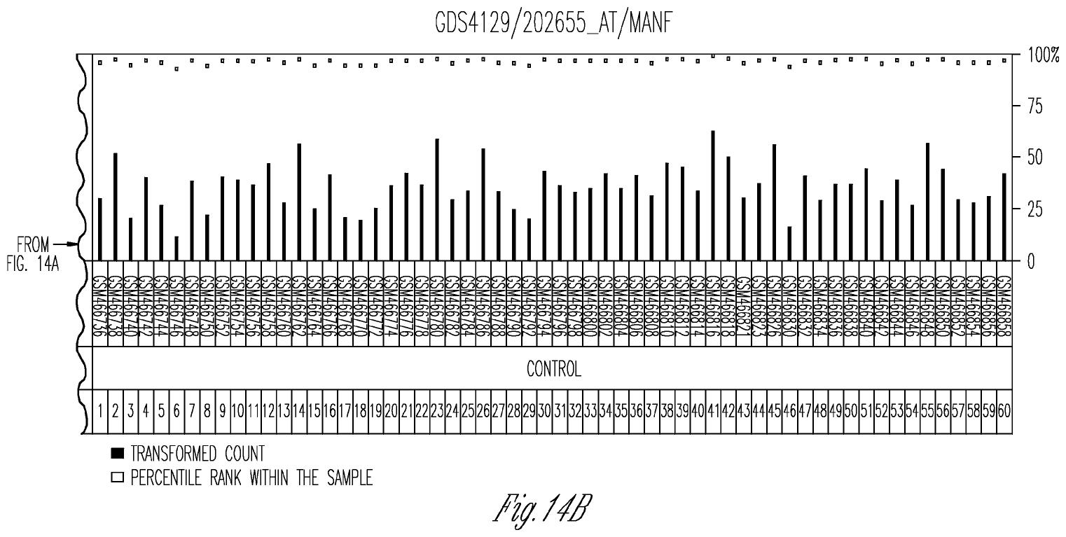

Data from the National Center for Biotechnology Information (NCBI) Gene Expression Omnibus (GEO) repository (available at: www.ncbi.nlm.nih.gov/geoprofiles/) was analyzed to determine MANF expression in immortalized B cells from 60 unrelated human individuals (Homo sapiens) treated in vitro with tunicamycin to induce ER stress (left) or untreated as control (right). ER stress induces the unfolded protein response (UPR). Immortalized B cells in which UPR was induced had higher levels of MANF expression (FIG. 14). These results provide insight into the molecular mechanisms underlying the UPR response.

### Example 5: MANF Expression in Mast Cells from Diabetic Prone Animals

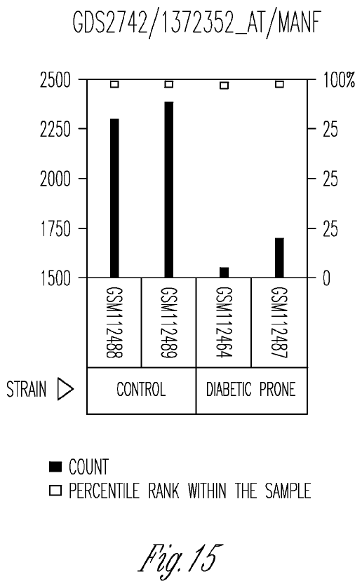

Data from the National Center for Biotechnology Information (NCBI) Gene Expression Omnibus (GEO) repository (available at: www.ncbi.nlm.nih.gov/geoprofiles/) was analyzed to determine MANF expression mast cells isolated from the pancreatic lymph nodes of 60 day old prediabetic BioBiobreeding (BB) DR lyp/lyp animals. The BB DR lyp/lyp strain is a model for type 1 diabetes mellitus (T1DM). The expression of MANF was higher in mast cells from control rats as compared to mast cells from rats that are diabetic prone (FIG. 15). Results provide insight into the role of mast cells in the pathogenesis of T1 DM.

### Example 6: CDNF Expression in Human Lymph Node Germinal Center

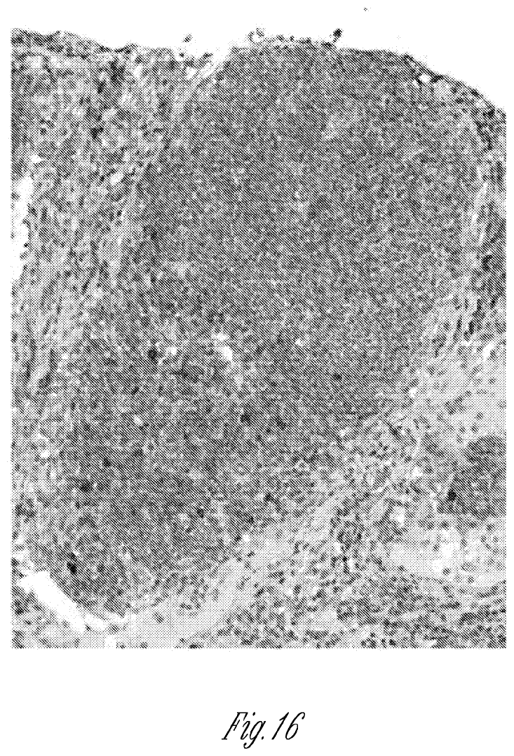

To obtain a general idea about CDNF expression in the immune system, the publically available Human Protein Atlas (http://www.proteinatlas.org) was surveyed. CDNF appears to be expressed in the light zone of the germinal center of the human lymph node (FIG. 16). This suggests that CDNF may play a role in B cell maturation.

### Example 7: Enhancing Vaccine Efficacy Using MANF Family Proteins as Vaccine Adjuvants

The demonstrated increased IgM antibody titer when MANF was co-injected with KLH antigen shows MANFs utility as a vaccine adjuvant. This example will further demonstrate the utility of MANF family proteins as vaccine adjuvants. Subjects will be administered immunological compositions comprising an antigen and an effective amount of a MANF family protein or fragment thereof (e.g., MANF or a fragment thereof, CDNF or a fragment thereof) as an adjuvant. The efficacy of the MANF family protein or fragment thereof as an adjuvant will be demonstrated by an enhanced immune response to the vaccine antigen. The enhanced immune response can be a more rapid development of antigen specific antibodies (e.g., IgM antibodies, IgG antibodies, or both). The enhanced immune response can be a higher titer of antigen specific antibodies. Control subjects will be administered only the antigen without the MANF family protein adjuvant.

### Example 8: Therapeutic Vaccines and Cancer Treatment Using MANF Family Proteins as Vaccine Adjuvants

The demonstrated increased IgM antibody titer when MANF was co-injected with KLH antigen shows MANFs utility as a vaccine adjuvant. This example will further demonstrate the utility of MANF family proteins as therapeutic vaccine adjuvants for the treatment of cancer. Subjects will be administered immunological compositions comprising a tumor rejection antigen and an effective amount a MANF family protein or fragment thereof (e.g., MANF or a fragment thereof, CDNF or a fragment thereof) as an adjuvant. The efficacy of the MANF family protein or fragment thereof as a therapeutic vaccine adjuvant will be demonstrated by an enhanced immune response to the tumor rejection antigen. The enhanced immune response can be a more rapid development of antigen specific antibodies (e.g., IgM antibodies, IgG antibodies, or both). The enhanced immune response can be a higher titer of antigen specific antibodies. Successful treatment will also be demonstrated by an improved therapeutic outcome (e.g., increased tumor shrinkage, increased probability of remission, longer period of remission, more rapid tumor shrinkage, etc.). Control subjects will be administered only the tumor rejection antigen without the MANF family protein adjuvant.

### Example 9: MANF Family Proteins as Adjuvants in Cancer Immunotherapy

The protective effect of MANF on T cells demonstrates that MANF may have utility in cell therapy applications. This example will further demonstrate the utility of MANF family proteins in enhancing CAR/T based immunotherapy for cancer treatment. Subjects will be treated with genetically engineered T cells that express a chimeric antigen receptor. The genetically engineered T cells can be cultured in the presence of MANF, primed with MANF 10 seconds to 1 hour before administration, and/or co-injected with MANF. The utility of MANF as a cell therapy adjuvant will be demonstrated by one or more of: increased survival of genetically engineered T cells (pre and/or post injection), increased tumor shrinkage, increased probability of remission, longer period of remission, or more rapid tumor shrinkage. Control subjects will be administered only the genetically engineered T cells without the MANF family protein adjuvant.

### Example 10: MANF Family Proteins as Cancer Immunotherapy Adjuvants

The protective effect of MANF on T-cells and the increased expression of MANF in activated cytotoxic T cell lines demonstrate that MANF may have utility in boosting immune responses. This example will further demonstrate the utility of MANF family proteins as adjuvants in cancer immunotherapy using bispecific monoclonal antibodies. The bispecific monoclonal antibodies will have at least two epitopes: one that binds to a tumor antigen and one that binds to a cytotoxic cell. The utility of MANF as a cancer immunotherapy adjuvant will be demonstrated by one or more of: increased tumor shrinkage, increased probability of remission, longer period of remission, or more rapid tumor shrinkage. Control subjects will be administered only the bispecific monoclonal antibodies without the MANF family protein adjuvant.

### Example 11: MANF Family Proteins as Bone Marrow Transplant Adjuvants

The protective effect of MANF on T cells demonstrates that MANF may have utility in cell therapy applications. This example will further demonstrate the utility of MANF family proteins in increasing the survival of bone marrow transplants. MANF family proteins can be utilized as adjuvants in the context of bone marrow transplants by administering MANF to the site of the bone marrow transplant before, concurrently, or after injection of the donor bone marrow. MANF family proteins can also be utilized as adjuvants in the context of bone marrow transplants by treating the donor bone marrow with a MANF family protein prior to transplantation of the donor bone marrow into the subject. The utility of MANF family proteins as bone marrow transplant adjuvants will be demonstrated by increased survival of transplanted bone marrow. Control subjects will undergo the bone marrow transplant without the use of MANF family proteins as an adjuvant.

### Example 12: Treatment of Chemotherapy or Radiation Therapy Side Effects with MANF Family Proteins

The protective effect of MANF on T cells and the induction of MANF expression in response to chemotherapeutic drugs demonstrate that MANF family proteins may have utility in treating or preventing side effects of chemotherapy and/or radiation therapy. This example will further demonstrate the utility of MANF family proteins in treating at least one condition associated with chemotherapy or radiation therapy. Subjects will be administered a MANF family protein to healthy tissue surrounding a tumor prior to or concurrently with administration of the chemo or radiation therapy. The utility of MANF family proteins in treating conditions associated with chemo or radiation therapy will be demonstrated by a decrease in healthy tissue loss in comparison to control subjects that did not receive the MANF family protein.

### Example 13: MANF Family Antagonist and Cancer Treatment

The protective effect of MANF on T cells treated with cell-death inducing drugs indicates that inhibition of MANF family protein activity may increase the efficacy of cancer treatment by sensitizing the tumor to chemotherapy or radiation therapy. Subjects will be administered a MANF family protein antagonist (e.g., function blocking antibodies or siRNA targeting expression of a MANF family protein) prior to or concurrently with chemo or radiation therapy. The utility of MANF family protein antagonists will be demonstrated by one or more of: increased tumor shrinkage, increased probability of remission, longer period of remission, or more rapid tumor shrinkage. Control subjects will be treated with only the chemo or radiation therapy.

### Example 14: MANF Family Antagonists and Treatment of Autoimmune Diseases

The protective effect of MANF on T cells, and the induction of MANF expression during T cell activation, suggests that inhibition of MANF family proteins may have utility in treatment of autoimmune or inflammatory disorders such as multiple sclerosis, irritable bowel syndrome, lupus, myasthenia gravis, rheumatoid arthritis, Hashimoto's thyroiditis, Grave's disease, autoimmune hepatitis, Alopecia, Addison's disease, Psoriasis, autoimmune pancreatitis, Celiac disease, pernicious anemia, Still's disease, juvenile arthritis, Felty syndrome, relapsing polychondritis, Guillain-Barré syndrome, vasculitis, or Crohn's disease. Subjects will be administered a MANF family protein antagonist (e.g., function blocking antibodies or siRNA targeting expression of a MANF family protein). The MANF family protein antagonist may be administered with the current standard of care for the indicated autoimmune or inflammatory disease. Control subjects will either be treated with a placebo or the standard of care without the MANF family protein antagonist.

### BIBLIOGRAPHY

Lowe D. A. and Urfer, R. (2013) A Review of the MANF Literature: Implications for the Development Program of Amarantus Bioscience Holdings Inc. http://content.stockpr.com/amarantus/files/pdf/MANF+WhitePaperf2013-04-30+FINAL.pdf
Petrova P S, Raibekas A, Pevsner J, Vigo N, Anafi M, Moore M K, Peaire A E, Shridhar V, Smith D I, Kelly J, Durocher Y, Commissiong J W (2003) MANF: a new mesencephalic, astrocyte-derived neurotrophic factor with selectivity for dopaminergic neurons. J Mol Neurosci 20:173-188.
Reed J C. (2006) Drug insight: cancer therapy strategies based on restoration of endogenous cell death mechanisms. Nat Clin Pract Oncol. 3:388-98.
Voutilainen M H, Bäck S, Pörsti E, Toppinen L, Lindgren L, Lindholm P, Pemen J, Saarma M, Tuominen R K. (2009) Mesencephalic astrocyte-derived neurotrophic factor is neurorestorative in rat model of Parkinson's disease. J Neurosci. 29:9651-9.
US Pub Appln No. 20090282495

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of enhancing an immunological response, the method comprising administering an immunogenic composition comprising an effective amount of a MANF family protein or fragments thereof and an antigen to a subject.

2. The method of claim 1, wherein the antigen is immunologically cross-reactive with a pathogen.

3. The method of claim 2, wherein the pathogen is a virus, a bacterium, a parasite, or a fungus.

4. The method of claim 1, wherein the antigen is immunologically cross-reactive with a cancer cell.

5. The method of claim 7, wherein the cancer cell is a prostate cancer cell, a breast cancer cell, a colorectal cancer cell, a lung cancer cell, a pancreatic cancer cell, a renal cancer cell, a melanoma cancer cell, an ovarian cancer cell, a B-cell malignancy cell, a leukemia cell, a lymphoma cell, a neuroblastoma cell, a glioblastoma cell, a skin cancer cell, a liver cancer cell, a testicular cancer cell, an adrenal cancer cell, esophageal cancer cell, a sarcoma, a gastrointestinal cancer cell, a cervical cancer cell, a bone cancer cell, or a combination thereof.

6. The method of claim 1, wherein the antigen is a tumor rejection antigen.

7. A method of treating cancer comprising administering an immunologic composition comprising an effective amount of a MANF family protein or fragment thereof and a tumor rejection antigen to a subject in need thereof.

8. The method of claim 7, wherein the tumor rejection antigen is a patient specific antigen, a tumor specific antigen or a tissue restricted antigen.

9. The method of claim 7, wherein the subject in need thereof has breast cancer, colorectal cancer, lung cancer, pancreatic cancer, renal cancer, melanoma cancer, ovarian cancer, a B-cell malignancy, leukemia, lymphoma, a neuroblastoma, a glioblastoma, skin cancer, a liver cancer, a testicular cancer, an adrenal cancer, esophageal cancer, a sarcoma, a gastrointestinal cancer, a cervical cancer, a bone cancer, or a combination thereof.

10. The method of claim 7, wherein the immunologic composition further comprises an aluminum-based salt, a squalene-oil-water emulsion, Bacillus Calmette-Guerin (BCG), or a combination thereof.

11. The method of claim 1, wherein the immunologic composition is administered by injection.

12. The method of claim 1, wherein the immunologic composition is administered by intranasal administration.

13. The method of claim 1, further comprising administering one or more booster compositions that comprise the antigen or tumor rejection antigen without the MANF family protein.

14. The method of claim 1, wherein the immunogenic composition is administered to the subject two or more times.

15. A method of treating cancer comprising administering to a subject in need thereof an effective amount of a MANF family protein or fragment thereof and genetically engineered T cells expressing a chimeric antigen receptor.

16. The method of claim 15, wherein the chimeric antigen receptor comprises an antigen recognition region and an endodomain.

17. The method of claim 16, wherein the target antigen of the antigen recognition region is α-Folate receptor, CAIX, CD19, CD20, CD22, CD30, CD33, CD44v7/8, CEA, EGP-2, EGP-40, erb-B2, erb-B 2,3,4, FBP, Fetal acetylcholine receptor, GD2, GD3, Her2/neu, IL-13R-a2, KDR, k-light chain, LeY, L1 cell adhesion molecule, MAGE-A1, Mesothelin, Murine CMV infected cells, MUC1, NKG2D ligands, Oncofetal antigen (h5T4), PSCA, PSMA, TAA targeted by mAb IgE, TAG-72, or VEGF-R2.

18. The method of claim 16, wherein the endodomain comprises ScFv-FcεRIγCAIX, ScFv-FcεRIγ, ScFv-CD3ζ (EBV), ScFv-CD3ζ, ScFv-CD28-CD3ζ, CD3ζ(EBV), ScFv-CD28-CD3ζ, CD3ζ, ScFv-CD3ζ, ScFv-41BB-CD3ζ, ScFv-41BB-CD3ζ, ScFv-CD3ζ (Influenza MP-1), ScFv-CD3ζ (VZV), ScFv-CD4-CD3ζ, CD3ζ/CD137/CD28, ScFv-CD28-41BB-CD3ζ, ScFv-CD8-CD3ζ, ScFv-FceRIγ, CD28/4-1BB-CD3ζ, ScFv-CD28-CD3ζ (Influenza), ScFv-CD28mut-CD3ζ, Heregulin-CD3ζ, ScFv-FcεRIγ (alloantigen), ScFv-CD28, ScFv-CD28-OX40-CD3ζ, ScFv-CD3ξ, IL-13-CD28-4-1 BB-CD3ζ, IL-13-CD3ζ, ScFv-CD28-CD3ζ, ScFV-CD4-FcεRIγ, ScFV-CD28-FcεRIγ, Ly49H-CD3ζ, NKG2D-CD3ζ, ScFV-CD3ζ, (vaccination), ScFv-b2c-CD3ζ, or FceRI-CD28-CD3ζ (+a-TAA IgE mAb).

19. The method of claim 15, wherein the genetically engineered T cells were grown in the presence of the MANF family protein or fragment thereof.

20. The method of claim 15, wherein the genetically engineered T cells were primed with the MANF family protein or fragment thereof.
